(19) United States
(12) Patent Application Publication
(10) Pub. No.: US 2007/0129297 A1
(43) Pub. Date: Jun. 7, 2007

(54) Treatment and prevention of asthma

(76) Inventor: Charles G. Cochrane, La Jolla, CA (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(21) Appl. No.: 11/445,494

(22) Filed: Jun. 1, 2006

Related U.S. Application Data
(63) Continuation of application No. PCT/US04/40665, filed on Dec. 3, 2004.
(60) Provisional application No. 60/526,787, filed on Dec. 4, 2003.

(51) Int. Cl. A61K 38/16; A61K 38/10; A61K 31/685; A61K 31/138

(52) U.S. Cl. 514/12; 514/13; 514/14; 514/15; 514/78; 514/651

(57) ABSTRACT

The invention provides compositions and methods for treating asthmatic conditions. Such compositions and methods utilize a lung surfactant mixture comprising a lung surfactant polypeptide.

### RELATED APPLICATIONS

This application is a continuation under 35 U.S.C. 111(a) of International Application No. PCT/US2004/040665 filed Dec. 3, 2004 and published in English as WO 2005/055994A1 on Jun. 23, 2005, which claimed priority from U.S. Provisional Application Ser. No. 60/256,787 filed Dec. 4, 2003; which applications and publication are incorporated herein by reference.

### FIELD OF THE INVENTION

The invention relates to pharmaceutical compositions and methods for preventing asthma that involve administration of a surfactant polypeptide.

### BACKGROUND OF THE INVENTION

Asthma is a chronic inflammatory disorder often characterized by airway inflammation and airway hyperreactivity (AHR). It is a leading cause of morbidity and mortality in children, adults, and the elderly. Current therapy for asthma includes treatment with bronchodilators, inhaled steroids, and leukotriene modifiers. Antigen specific immune therapy has also been used to desensitize patients to specific allergens. However, such desensitization can be ineffective for many allergic asthmatics sensitive to multiple antigens. Similarly, inhaled corticosteroids have severe adverse effects along with suppression of Th1 and Th2 cytokine responses. Moreover, even with currently available therapies, the incidence of asthma has continued to increase over the last two decades.

Thus, new asthmatic therapeutic agents are needed that are more effective but have fewer adverse effects.

### SUMMARY OF THE INVENTION

The invention generally relates to compositions and methods for treating asthmatic conditions.

The compositions of the invention include at least one lung surfactant polypeptide. The lung surfactant polypeptide can have about 10 to about 60 amino acid residues with an amino acid sequence of alternating hydrophobic and hydrophilic amino acid residue regions represented by the formula (ZₐU_{b})_{c}Z_{d}, where Z is a hydrophilic amino acid residue, U is a hydrophobic amino acid residue, “a” is an integer of about 1 to about 5, “b” is an integer of about 3 to about 20, “c” is an integer of about 1 to about 10, and “d” is an integer of about 0 to about 3.

In exemplary lung-surfactant polypeptides, Z is histidine, lysine, arginine, aspartic acid, glutamic acid, 5-hydroxylysine, 4-hydroxyproline, and/or 3-hydroxyproline, and U is valine, isoleucine, leucine, cysteine, tyrosine, phenylalanine, and/or an α-aminoaliphatic carboxylic acid, such as α-aminobutanoic acid, α-aminopentanoic acid, α-amino-2-methylpropanoic acid, or α-aminohexanoic acid.

In one embodiment, the lung surfactant polypeptide can be a polypeptide of the following structure:

(SEQ ID NO:18)

(Xa)(Xb)LLLL(Xa)LLLL(Xa)(Xb)LLLL(Xa)LLL(Xa)(Xb)

wherein each Xa is separately selected from lysine or arginine, and each Xb is separately selected from aspartic acid or glutamic acid.

In some embodiments, the surfactant proteins have any one of the following sequences, or a combination thereof:

| | |
|---|---|
| KLLLLKLLLLKLLLLKLLLLK, | (SEQ ID NO:1) |
| KLLLLLLLLKLLLLLLLLKLL, | (SEQ ID NO:2) |
| KKLLLLLLLKKLLLLLLLKKL, | (SEQ ID NO:3) |
| DLLLLDLLLLDLLLLDLLLLD, | (SEQ ID NO:4) |
| RLLLLRLLLLRLLLLRLLLLR, | (SEQ ID NO:5) |
| RLLLLLLLLRLLLLLLLLRLL, | (SEQ ID NO:6) |
| RRLLLLLLLRRLLLLLLLRRL, | (SEQ ID NO:7) |
| RLLLLCLLLRLLLLCLLLR, | (SEQ ID NO:8) |
| RLLLLCLLLRLLLLCLLLRLL, | (SEQ ID NO:9) |
| RLLLLCLLLRLLLLCLLLRLLLLCLLLR, | (SEQ ID NO:10) |
| or | |
| HLLLLHLLLLHLLLLHLLLLH. | (SEQ ID NO:11) |

The compositions for pulmonary administration can contain a surfactant mixture of (i) 50-95 dry weight percent phospholipid, (ii) 2-25 dry weight percent of a spreading agent effective to promote incorporation and distribution of the phospholipid within the surface lining layer of the lung, and (iii) 0.1 to 10 dry weight percent of lung-surfactant polypeptide.

In specific exemplary embodiments, the phospholipid of the surfactant mixture includes dipalmitoyl phosphatidylcholine (DPPC) and palmitoyl, oleoyl phosphatidylglycerol (POPG) in a mole ratio of between 4:1 and 2:1. An exemplary spreading agent is a fatty acid or fatty alcohol having a fatty acyl chain length of at least 10 carbon atoms, such as palmitic acid or cetyl alcohol.

The surfactant compositions of the invention can be inhaled or administered as an aerosol. Where the aerosol particles are formed from a liquid dispersion, the surfactant formulation may be dispersed in aqueous aerosol droplets. Where the particles are in the form of a dry powder, the particles are dehydrated, or substantially dehydrated. The aerosol particles can have a mass median aerodynamic diameter in the 1-5 μm size range.

In some embodiments, the compositions of the invention may be administered as a liquid, for example, by liquid bolus administration.

The invention also provides a method for treating asthma in a mammal comprising administering to the mammal a therapeutically effective amount of a composition comprising a lung surfactant polypeptide of the invention. One of skill in the art will often choose to administer the composition directly to pulmonary tissues (e.g. by inhaler, through the use of a nebulizer or as an aerosol). The asthmatic condition treated by the present methods can be, for example, acute inflammatory asthma, allergic asthma, iatrogenic asthma and related asthmatic conditions.

In another aspect, the invention includes a method of administering a lung surfactant polypeptide to a patient. Administration can be by inhalation. The method includes generating a surfactant mixture composed of (i) 50-95 dry weight percent phospholipid, (ii) 2-25 dry weight percent of a spreading agent effective to promote incorporation and distribution of the phospholipid within the surface-lining layer of the lung, and (iii) 0.1 to 10 dry weight percent of lung-surfactant polypeptide. The lung surfactant polypeptide can be a polypeptide having between 10-60 amino acid residues and has an amino acid sequence of alternating hydrophobic and hydrophilic amino acid residue regions.

For example, the lung surfactant polypeptide can be represented by the formula (ZₐU_{b})_{c}Z_{d}, where Z is a hydrophilic amino acid residue, U is a hydrophobic amino acid residue, “a” has an average value of 1-5, “b” has an average value of 3-20, “c” is 1-10, and “d” is 0 to 3. The resulting formulation contains 1-80, or 2-50 dry weight percent of the active agent.

The formulation can be converted to a particle composition whose particles have a mass median aerodynamic diameter in the 1-5 μm. The particles are administered in the form of an aerosol composition to the respiratory tract of the patient, in a therapeutically effective amount.

In some embodiments, the formulation is prepared by dissolving or dispersing the lung surfactant and other components of the formulation in a solvent, which may be an aqueous, organic, or mixed solvent. The formulation can be converted to a particle composition for aerosol administration by spray drying the mixture under conditions effective to produce dry particles having the desired 1-5 μm MMAD size range. In other embodiments, the formulation can be converted to a particle composition for aerosol administration by lyophilizing a liquid composition to dryness, and comminuting the dried mixture to form dry particles of the desired size range.

Liquid or dry particles can be administered by inhalation in aerosol form. The formulation may also be in an aqueous dispersion form, e.g., a liposomal dispersion, which is aerosolized to form liquid droplets having dispersed formulation particles dispersed therein.

These and other objects and features of the invention will become more fully apparent in view of the following description of the invention.

### DETAILED DESCRIPTION OF THE INVENTION

The invention relates to compositions and methods for treating or preventing asthma that have at least one lung surfactant polypeptide. Other ingredients can be included to facilitate delivery and dispersion of the composition within the lung, for example, phospholipids and spreading agents.

Definitions

The terms below have the following meanings, unless indicated otherwise.

“Amino acid” refers to refers to amino acid residues that can be linked together through formation of a covalent bond between an amino group and a carboxyl group. For example, amino acids can make up a polypeptide or protein. Both genetically-encoded and non-genetically-encoded amino acids are contemplated. Genetically-encoded amino acids are commonly in the natural L-form. However, D-amino acids, substituted amino acids (e.g., amino acids with modified side chain groups) amino acid metabolites and catabolites, amino acids with “retro” backbones, and amino acid mimics or analogs are also contemplated for use in—and are thus encompassed by—the present invention. In keeping with standard polypeptide nomenclature, J. Biol. Chem., 243:3557-59, 1969, abbreviations for the more common amino acid residues are as shown in the following Table of Correspondence.

Table of Correspondence

Symbol

| 1-Letter | 3-Letter | Amino Acid |
|---|---|---|
| Y | Tyr | L-tyrosine |
| G | Gly | Glycine |
| F | Phe | L-phenylalanine |
| M | Met | L-methionine |
| A | Ala | L-alanine |
| S | Ser | L-serine |
| I | Ile | L-isoleucine |
| L | Leu | L-leucine |
| T | Thr | L-threonine |
| V | Val | L-valine |
| P | Pro | L-proline |
| K | Lys | L-lysine |
| H | His | L-histidine |
| Q | Gln | L-glutamine |
| E | Glu | L-glutamic acid |
| W | Trp | L-tryptophan |
| R | Arg | L-arginine |
| D | Asp | L-aspartic acid |
| N | Asn | L-asparagine |
| C | Cys | L-cysteine |
| X | Xaa | Unknown/other |

It should be noted that, unless otherwise indicated, the amino acid residue sequences represented herein by formulae have a left to right orientation in the conventional direction of amino-terminus to carboxy-terminus. In addition, the phrase “amino acid residue” is broadly defined to include the amino acids listed in the Table of Correspondence and modified and unusual amino acids, such as those listed in 37 C.F.R. §1.822(b)(4), and incorporated herein by reference. The phrase “amino acid residue” is also broadly defined to include non-genetically-encoded amino acids, D-amino acids, substituted amino acids (e.g., amino acids with modified side chain groups), modified amino acids (e.g., amino acid metabolites, catabolites, and amino acids with “designed” side chains), and amino acid mimics or analogs.

Furthermore, it should be noted that a dash at the beginning or end of an amino acid residue sequence generally indicates a bond to a radical such as H and OH (hydrogen and hydroxyl) at the amino- and carboxy-termini, respectively, or a further sequence of one or more amino acid residues. In addition, it should be noted that a virgule (/) at the right hand end of a residue sequence indicates that the sequence is continued on the next line.

“Human” means that a material causes substantially no immune reaction in a human. For example, the lung surfactant polypeptides of the invention may not all be derived from a human source or may not have an amino acid sequence identical to known human lung proteins, but such lung surfactant polypeptides may be referred to as “human” so long as they cause substantially no immune response in a human.

“Isolated” means that the isolated material has been removed from its natural environment. In some embodiments, an “isolated” material may be present in a composition or another environment where it would not be naturally found. For example, a lung surfactant polypeptide of the invention may be isolated even though it has been mixed into a composition containing other ingredients or is present in a recombinant organism that is used for recombinant production of the polypeptide.

“Pharmaceutically acceptable” is a term that refers to molecular entities and compositions that do not produce an allergic or similar untoward reaction when administered to a human.

A “protein” or “polypeptide” or “peptide” is a biopolymer composed of amino acid or amino acid analog subunits, typically some or all of the 20 common L-amino acids found in biological proteins, linked by peptide intersubunit linkages, or other intersubunit linkages that are consistent with enzyme-substrate or receptor binding ligand interactions. The protein has a primary structure represented by its subunit sequence, and may have secondary helical or pleat structures, as well as overall three-dimensional structure. Although “protein” commonly refers to a relatively large polypeptide, e.g., containing 30 or more amino acids, and “peptide” to or “polypeptide” to smaller polypeptides, the terms are also used interchangeably herein. That is, the term “protein” may refer to a larger polypeptide, e.g., greater than 30 amino acids, but does not necessarily exclude a smaller polypeptide, and the term “polypeptide” may refer to a smaller peptide, e.g., fewer than 30 amino acids, but may also include larger proteins.

“Purified” means that a material has been removed from the environment in which it was made. A material may be partially or substantially purified and need not be completely (100%) pure. For example, a lung surfactant polypeptide of the invention may be purified after it has been chemically or recombinantly synthesized by removing some or all of the unreacted chemicals, side products, cellular debris and other components.

“Surfactant activity” refers to the ability of any substance, such as an organic molecule, protein or polypeptide, when combined with lipids, either alone or in combination with other organic molecules, to lower surface tension at an air/water interface. The measurement can be made with a Wilhelmy balance or pulsating bubble surfactometer by an in vitro assay. See, for example that of King et al, Am. J. Physiol. 223:715-726 (1972), or the assay illustrated herein, which utilizes a measurement of surface tension at an air-water interface when a protein or polypeptide is admixed with a phospholipid. In addition, in vivo measurements of increases in compliance or airflow at a given pressure of air entering the lung can be readily made, such as in the assay of Robertson, Lung, 158:57-68 (1980). In this assay, the sample to be assessed is administered through an endotracheal tube to fetal rabbits or lambs delivered prematurely by Caesarian section. (These “preemies” lack their own pulmonary surfactant, and are supported on a ventilator). Measurements of lung compliance, blood gases and ventilator pressure provide indices of activity. In vitro assays of surfactant activity, which is assessed as the ability to lower the surface tension of a pulsating bubble, and in vivo assays utilizing fetal rabbits, are described in detail by Revak et al, Am. Rev. Respir. Dis., 134:1258-1265 (1986).

“Surfactant molecule” refers to organic molecules having surfactant activities and when admixed with pharmaceutically acceptable lipids form a surfactant that has greater surfactant activity than the lipids alone as evidenced by the lower ΔP values.

“Natural pulmonary surfactant” refers to a pulmonary surfactant (PS) that lines the alveolar epithelium of mature mammalian lungs. Natural or native PS has been described as a “lipoprotein complex” because it contains both phospholipids and apoproteins that interact to reduce surface tension at the lung air-liquid interface. Natural surfactant contains several lipid species of which dipalmitoyl phosphatidylcholine (DPPC) is the major component. At least four proteins are typically present in natural pulmonary surfactants, SP-A, SP-B, SP-C and SP-D. Of these four, SP-B and SP-C are distinct, low molecular weight, relatively hydrophobic proteins that have been shown to enhance the surface-active properties of surfactant phospholipid mixtures, presumably by facilitating transfer of lipids from the bulk phase lamellar organization to the air-water interface and also by stabilizing the lipid monolayer during expiration. The structure of SP-B is unusual in that charged amino acids (predominantly basic) are located at fairly regular intervals within stretches of otherwise hydrophobic residues. For the domain consisting of residues 59-80 of the native SP-B sequence, these charged groups have been shown to be necessary for biological activity. In addition, natural and synthetic peptides, which are modeled on this hydrophobic-hydrophilic domain when combined with DPPC and PG, exhibit good surfactant activity.

Natural surfactant protein is stored in lung epithelial cells in the form of lamellar bodies and, following export, it undergoes a structural transition to form tubular myelin before giving rise to a monolayer at the air-water interface. It has been proposed that surfactant proteins SP-A, SP-B and SP-C may facilitate these structural transitions and stabilize the lipid monolayer during expansion and contraction of the alveolus; however, a complete understanding of lipid-protein interactions at the molecular level is presently lacking.

“Pulmonary administration” refers to any mode of administration that delivers a pharmaceutically active substance to any surface of the lung. The modes of delivery can include, but are not limited to, those suitable for inhalation as a liquid suspension, as a dry powder “dust” or insufflate, or as an aerosol.

“Phospholipids” refers to amphipathic lipids that are composed of a nonpolar hydrophobic tail, a glycerol or sphingosine moiety, and a polar head. The nonpolar hydrophobic tail is usually a saturated or unsaturated fatty acid group. The polar head has a phosphate group that is often attached to a nitrogen-containing base.

“Spreading agent” means a compound that promotes incorporation and distribution of phospholipid(s) within the surface lining layer of the lungs, that is, promotes the spreading of phospholipids at the air/liquid interface at the surface lining layer of the lungs.

“Aerodynamic diameter” is defined as the diameter of an equivalent spherical particle of unit density that has the same settling velocity as the characterized particle. That is, regardless of the shape or size of particle, the particle is imagined to be transformed into a sphere of unit density. The diameter of that sphere is the aerodynamic diameter. Thus, particles having aerodynamic diameters in the 1-5 micron size have the same aerodynamic properties as spherical particles of unit density having diameters in the 1-5 micron size range. The aerodynamic properties of particles can be measured experimentally using conventional techniques such as cascade impaction, elutriators or sedimentation cells. Often the measuring technique used is one that most closely resembles the situation in which the aerosol is being employed.

“Mass median aerodynamic diameter” of a collection of particles refers to the median aerodynamic diameter (MMAD) of the mass of the particles. That is, half of the mass of the particles is at or below the MMAD, and half above. The heterodispersity of aerosol particles can be defined by a geometric standard deviation (GSD). If all of the particles are the same size and shape, the GSD is 1. A GSD of 3.5 indicates a highly heterodisperse collection of particles. Preferably aerosol particles of the present invention are formed under conditions that give a GSD of between 1 and 3, preferably 1-2.

“Model surfactant mixture” or “Surfaxin®” refers to a surfactant mixture prepared in accordance with the present invention, using the surfactant-mixture components set out in Examples 1 and 2.

Lung Surfactant Polypeptides

The lung surfactant polypeptides employed in the invention are polypeptides that include amino acid residue sequences having alternating charged and uncharged amino acid residue regions. Polypeptide surfactants having amino acid residue sequences with alternating hydrophobic and hydrophilic amino acid residue regions are also employed in the compositions and methods of the present invention. Lung surfactant polypeptides can have at least about 4, or at least about 8, or at least about 10, amino acid residues. Such lung surfactant polypeptides are generally not more than about 60 amino acid residues in length, although longer and even full-length native lung surfactant proteins are also contemplated. Examples of lung surfactant polypeptides that can be used in the compositions and methods of the invention are described in U.S. Pat. Nos. 6,013,619, 5,789,381, 5,407,914, 5,260,273 and 5,164,369, all of which are incorporated by reference herein.

Lung surfactant polypeptides of the present invention can have alternating groupings of charged and uncharged amino acid residues amino acid residues as represented by the formula [(Charged)ₐ (Uncharged)_{b}]_{c} (Charged)_{d}, wherein “a” has an average value of about 1 to about 5; “b” has an average value of about 3 to about 20; “c” is 1 to 10; and “d” is 0 to 3. Organic surfactant molecules not comprised solely of amino acid residues alone preferably have a similar structure constituted by alternating groupings of charged and uncharged (or hydrophilic/hydrophobic) constituent molecules.

As known to one of skill in the art, amino acids can be placed into different classes depending primarily upon the chemical and physical properties of the amino acid side chain. For example, some amino acids can be charged, hydrophilic or polar amino acids and others can be uncharged, hydrophobic or nonpolar amino acids. Polar amino acids include amino acids having acidic, basic or hydrophilic side chains and nonpolar amino acids include amino acids having aromatic or hydrophobic side chains. Nonpolar amino acids may be further subdivided to include, among others, aliphatic amino acids. The definitions of the classes of amino acids as used herein are as follows:

“Nonpolar Amino Acid” refers to an amino acid having a side chain that is uncharged at physiological pH, that is not polar and that is generally repelled by aqueous solution. Examples of genetically encoded hydrophobic amino acids include alanine, leucine, isoleucine, methionine, phenylalanine, tryptophan, tyrosine and valine. In some embodiments, cysteine is a nonpolar amino acid. Examples of non-genetically encoded nonpolar amino acids include t-BuA, Cha, norleucine, and/or an α-aminoaliphatic carboxylic acid, such as α-aminobutanoic acid, α-aminopentanoic acid, α-amino-2-methylpropanoic acid, or α-aminohexanoic acid.

“Aromatic amino acid” refers to a nonpolar amino acid having a side chain containing at least one ring having a conjugated π-electron system (aromatic group). The aromatic group may be further substituted with substituent groups such as alkyl, alkenyl, alkynyl, hydroxyl, sulfonyl, nitro and amino groups, as well as others. Examples of genetically encoded aromatic amino acids include phenylalanine, tyrosine and tryptophan. Commonly encountered non-genetically encoded aromatic amino acids include phenylglycine, 2-naphthylalanine, β-2-thienylalanine, 1,2,3,4-tetrahydroisoquinoline-3-carboxylic acid, 4-chlorophenylalanine, 2-fluorophenylalanine, 3-fluorophenylalanine and 4-fluorophenylalanine.

“Aliphatic amino acid” refers to a nonpolar, uncharged amino acid having a saturated or unsaturated straight chain, branched or cyclic hydrocarbon side chain. Examples of genetically encoded aliphatic amino acids include Ala, Leu, Val and Ile. Examples of non-encoded aliphatic amino acids include Nle.

“Polar amino acid” refers to a hydrophilic amino acid having a side chain that is charged or uncharged at physiological pH and that has a bond in which the pair of electrons shared in common by two atoms is held more closely by one of the atoms. Polar amino acids are generally hydrophilic, meaning that they have an amino acid having a side chain that is attracted by aqueous solution. Examples of genetically encoded polar amino acids include asparagine, glutamine, lysine and serine. In some embodiments, cysteine is a polar amino acid. Examples of non-genetically encoded polar amino acids include citrulline, homocysteine, N-acetyl lysine and methionine sulfoxide.

“Acidic Amino Acid” refers to a hydrophilic amino acid having a side chain pK value of less than 7. Acidic amino acids typically have negatively charged side chains at physiological pH due to loss of a hydrogen ion. Examples of genetically encoded acidic amino acids include aspartic acid (aspartate) and glutamic acid (glutamate).

“Basic Amino Acid” refers to a hydrophilic amino acid having a side chain pK value of greater than 7. Basic amino acids typically have positively charged side chains at physiological pH due to association with hydronium ion. Examples of genetically encoded basic amino acids include arginine, lysine and histidine. Examples of non-genetically encoded basic amino acids include the non-cyclic amino acids ornithine, 2,3-diaminopropionic acid, 2,4-diaminobutyric acid and homoarginine.

“Ionizable Amino Acid” or “Charged Amino Acid” refers to an amino acid that can be charged at a physiological pH. Such ionizable or charges amino acids include acidic and basic amino acids, for example, D-aspartic acid, D-glutamic acid, D-histidine, D-arginine, D-lysine, D-hydroxylysine, D-ornithine, D-3-hydroxyproline, L-aspartic acid, L-glutamic acid, L-histidine, L-arginine, L-lysine, L-hydroxylysine, L-ornithine or L-3-hydroxyproline.

As will be appreciated by those having skill in the art, the above classifications are not absolute. Several amino acids exhibit more than one characteristic property, and can therefore be included in more than one category. For example, tyrosine has both a nonpolar aromatic ring and a polar hydroxyl group. Thus, tyrosine has several characteristics that could be described as nonpolar, aromatic and polar. However, the nonpolar ring is dominant and so tyrosine is generally considered to be nonpolar. Similarly, in addition to being able to form disulfide linkages, cysteine also has nonpolar character. Thus, while not strictly classified as a hydrophobic or nonpolar amino acid, in many instances cysteine can be used to confer hydrophobicity or nonpolarity to a peptide.

The classifications of the above-described genetically encoded and non-encoded amino are for illustrative purposes only and do not purport to be an exhaustive list of amino acid residues that may comprise the lung surfactant polypeptides described herein. Other amino acid residues that are useful for making the lung surfactant polypeptides described herein can be found, e.g., in Fasman, 1989, CRC Practical Handbook of Biochemistry and Molecular Biology, CRC Press, Inc., and the references cited therein. Another source of amino acid residues is provided by the website of RSP Amino Acids Analogues, Inc. (www.amino-acids.com). Amino acids not specifically mentioned herein can be conveniently classified into the above-described categories on the basis of known behavior and/or their characteristic chemical and/or physical properties as compared with amino acids specifically identified.

In some embodiments, surfactant polypeptides include a sequence having alternating groupings of amino acid residues as represented by the formula (ZₐU_{b})_{c}Z_{d}, wherein Z is a charged amino acid and U is an uncharged amino acid; “a” has an average value of about 1 to about 5; “b” has an average value of about 3 to about 20, “c” is 1 to 10; and “d” is 0 to 3.

In some embodiments, Z is histidine, lysine, arginine, aspartic acid, glutamic acid, 5-hydroxylysine, 4-hydroxyproline, and/or 3-hydroxyproline, and U is valine, isoleucine, leucine, cysteine, tyrosine, phenylalanine, and/or an α-aminoaliphatic carboxylic acid, such as α-aminobutanoic acid, α-aminopentanoic acid, α-amino-2-methylpropanoic acid, or α-aminohexanoic acid.

In another embodiment, polypeptides of the present invention have alternating groupings or amino acids residue regions as represented by the formula (BₐU_{b})_{c}B_{d}, wherein B is an amino acid residue independently selected from the group consisting of histidine, lysine, 5-hydroxylysine, 4-hydroxyproline, and 3-hydroxyproline; and U is an amino acid residue independently selected from the group consisting of valine, isoleucine, leucine, cysteine, tyrosine, and phenylalanine. In one variation, B is an amino acid derived from collagen and is selected from the group consisting of 5-hydroxylysine, 4-hydroxyproline, and 3-hydroxyproline; “a” has an average value of about 1 to about 5; “b” has an average value of about 3 to about 20; “c” is 1 to 10; and “d” is 0 to 3.

In still another embodiment, surfactant polypeptides of the present invention include a sequence having alternating groupings of amino acid residues as represented by the formula (BₐJ_{b})_{c}B_{d}, wherein B is an amino acid residue independently selected from the group consisting of histidine, 5-hydroxylysine, 4-hydroxyproline, and 3-hydroxyproline; and J is an α-aminoaliphatic carboxylic acid; “a” has an average value of about 1 to about 5; “b” has an average value of about 3 to about 20; “c” is 1 to 10; and “d” is 0 to 3.

In various embodiments including “J” in the relevant formula, J is an α-aminoaliphatic carboxylic acid having four to six carbons, inclusive. In other embodiments, J is an α-aminoaliphatic carboxylic acid having six or more carbons, inclusive. In yet other variations, J is preferably selected from the group consisting of α-aminobutanoic acid, α-aminopentanoic acid, α-amino-2-methylpropanoic acid, and α-aminohexanoic acid.

Another embodiment contains surfactant polypeptides including a sequence having alternating groupings of amino acid residues as represented by the formula (ZₐU_{b})_{c}Z_{d}, wherein Z is an amino acid residue independently selected from the group consisting of R, D, E, and K; and U is an amino acid residue independently selected from the group consisting of V, I, L, C, Y and F. In some embodiments, U is selected from the group consisting of V, I, L, C and F; or from the group consisting of L and C. The integer “a” has an average value of about 1 to about 5; “b” has an average value of about 3 to about 20; “c” is 1 to 10; and “d” is 0 to 3.

In the foregoing formulae, Z and U, Z and J, D and U, and B and J are amino acid residues that, at each occurrence, are independently selected. In addition, in each of the aforementioned formulae, “a” generally has an average value of about 1 to about 5; “b” generally has an average value of about 3 to about 20; “c” is 1 to 10; and “d” is 0 to 3.

In one variation of the foregoing embodiments, Z and B are charged amino acid residues. In other embodiments, Z and B are hydrophilic or positively charged amino acid residues. In one variation, Z is selected from the group consisting of R, D, E and K. In another embodiment, Z is preferably selected from the group consisting of R and K. In yet another, B is selected from the group consisting of histidine, 5-hydroxylysine, 4-hydroxyproline, and 3-hydroxyproline. In another embodiment, B is a collagen constituent amino acid residue and is selected from the group consisting of 5-hydroxylysine, (δ-hydroxylysine), 4-hydroxyproline, and 3-hydroxyproline. In another embodiment, B is histidine.

In various disclosed embodiments, U and J are uncharged amino acid residues. In some embodiments, U and J are hydrophobic amino acid residues. For example, in some embodiments, U is selected from the group consisting of V, I, L, C, Y, and F. In another embodiment, U is selected from the group consisting of V, I, L, C, and F. In yet another embodiment, U is selected from the group consisting of L and C. In various embodiments, U is L.

Similarly, in various embodiments, B is an amino acid selected from the group consisting of histidine, 5-hydroxylysine, 4-hydroxyproline, and 3-hydroxyproline. Alternatively, B may be selected from the group consisting of collagen-derived amino acids, which includes 5-hydroxylysine, 4-hydroxyproline, and 3-hydroxyproline.

In another embodiment of the present invention, charged and uncharged amino acids are selected from groups of modified amino acids. For example, in one embodiment, a charged amino acid is selected from the group consisting of citrulline, homoarginine, or ornithine, to name a few examples. Similarly, in various preferred embodiments, the uncharged amino acid is selected from the group consisting of α-aminobutanoic acid, α-aminopentanoic acid, α-amino-2-methylpropanoic acid, and α-aminohexanoic acid.

In various embodiments of the present invention, variables “a”, “b”, “c” and “d” are integers that indicate the number of charged or uncharged residues (or hydrophilic or hydrophobic residues).

In some embodiments, “a” has an average value of about 1 to about 5, or of about 1 to about 3, or of about 1 to about 2, or of about 1.

In various embodiments, “b” is an integer with an average value of about 3 to about 20, or about 3 to about 12, or about 3 to about 10, or about 4 to about 8. In one embodiment, “b” is about 4.

In various embodiments, “c” is an integer with an average value of about 1 to about 10, or about 2 to about 10, or about 3 to about 8, or about 4 to about 8, or about 3 to about 6. In one embodiment, “c” is about 4.

In various embodiments, “d” is an integer with an average value of about 0 to about 3 or about 1 to about 3. In one embodiment, “d” is about 0 to about 2, or 1 to 2; in another embodiment, “d” is 1.

By stating that an amino acid residue—e.g., a residue represented by Z or U—is independently selected, it is meant that at each occurrence, a residue from the specified group is selected. That is, when “a” is 2, for example, each of the hydrophilic residues represented by Z will be independently selected and thus can include, for example, RR, RD, RE, RK, DR, DD, DE, DK, etc.

By stating that “a” and “b” have average values, it is meant that although the number of residues within the repeating sequence (e.g., ZₐU_{b}) can vary somewhat within the peptide sequence, the average values of “a” and “b” would be about 1 to about 5 and about 3 to about 20, respectively. For example, using the formula (ZₐU_{b})_{c}Z_{d} for the peptide designated “KL8” in Table 1 below, the formula can be rewritten as K₁L₈K₁L₈K₁L₂, wherein the average value of “b” is six [i.e., (8+8+2)/3=6], “c” is three and “d” is zero.

One example of a lung surfactant polypeptide that can be used in the compositions and methods of the invention is SEQ ID NO:18.

(Xa)(Xb)LLLL(Xa)LLLL(Xa)(Xb)LLLL(Xa)LLL(Xa)(Xb) (SEQ ID NO:18)

wherein each Xa is separately selected from lysine or arginine, and each Xb is separately selected from aspartic acid or glutamic acid.

Other exemplary preferred polypeptides of the invention are shown in Table 1 below.

TABLE 1

| | SEQ | |
|---|---|---|
| Designation¹ | ID NO | Amino Acid Residue Sequence |
| KL4 | 1 | KLLLLKLLLLKLLLLKLLLLK |
| KL8 | 2 | KLLLLLLLLKLLLLLLLLKLL |
| KL7 | 3 | KKLLLLLLLKKLLLLLLLKKL |
| DL4 | 4 | DLLLLDLLLLDLLLLDLLLLD |
| RL4 | 5 | RLLLLRLLLLRLLLLRLLLLR |
| RL8 | 6 | RLLLLLLLLRLLLLLLLLRLL |
| RL7 | 7 | RRLLLLLLLRRLLLLLLLRRL |
| RCL1 | 8 | RLLLLCLLLRLLLLCLLLR |
| RCL2 | 9 | RLLLLCLLLRLLLLCLLLRLL |
| RCL3 | 10 | RLLLLCLLLRLLLLCLLLRLLLLCLLLR |
| HL4 | 11 | HLLLLHLLLLHLLLLHLLLLH |

¹The designation is an abbreviation for the indicated amino acid residue sequence.

Also suitable are composite polypeptides of about 4 to 60 amino acid residues having a configuration that maximizes their interaction with the alveoli. A composite polypeptide consists essentially of an amino terminal sequence and a carboxy terminal sequence. The amino terminal sequence has an amino acid sequence of a hydrophobic region polypeptide or a hydrophobic peptide of this invention, preferably hydrophobic polypeptide, as defined in the above formula. The carboxy terminal sequence has the amino acid residue sequence of a subject carboxy terminal peptide.

Proteins and polypeptides derived from or having characteristics similar to those of natural Surfactant Protein (SP) are useful in the present methods. As noted, SP isolated from any mammalian species may be utilized, although bovine, porcine and human surfactants are particularly preferred.

Natural surfactant proteins include SP-A, SP-B, SP-C or SP-D, or fragments thereof, alone or in combination with lipids. A preferred fragment is the amino-terminal residues 1-25 of SP-B.

Many amino acid sequences related to such natural surfactant proteins can be found in the NCBI database. For example, a sequence of human pulmonary surfactant associated protein A1 can be found in the NCBI database as accession number NP 005402 (gi: 13346504). See website at ncbi.nlm.nih.gov. This sequence for human SP-A1 is provided below as follows (SEQ ID NO:12).

| | |
|---|---|
| 1 | MWLCPLALNL ILMAASGAVC EVKDVCVGSP GIPGTPGSHC |
| 41 | LPGRHGRDGL KGDLGPPGPM GPPGEMPCPP GNDGLPGAPG |
| 81 | IPGECGEKGE PGERGPPGLR ARLDEELQAT LHDFRHQILQ |
| 121 | TRGALSLQGS IMTVGEKVFS SNGQSITFDA IQEACARAGG |
| 161 | RIAVPRNPEE NEAIASFVKK YNTYAYVGLT EGPSPGDFRY |
| 201 | SDGTPVNYTN WYRGEPAGRG KEQCVEMYTD GQWNDRNCLY |
| 241 | SRLTICEF |

An amino acid sequence for human pulmonary surfactant associated protein A2 can be found in the NCBI database as accession number NP 008857 (gi: 13346506). See website at ncbi.nlm.nih.gov. This sequence for human SP-A2 is provided below as follows (SEQ ID NO:13).

| | |
|---|---|
| 1 | MWLCPLALNL ILMAASGAAC EVKDVCVGSP GIPGTPGSHG |
| 41 | LPGRDGRDGV KGDPGPPGPM GPPGETPCPP GNNGLPGAPG |
| 81 | VPGERGEKGE AGERGPPGLP AHLDEELQAT LHDFRHQILQ |
| 121 | TRGALSLQGS IMTVGEKVFS SNGQSITFDA IQEACARAGG |
| 161 | RIAVPRNPEE NEAIASFVKK YNTYAYVGLT EGPSPGDFRY |
| 201 | SDGTPVNYTN WYRGEPAGRG KEQCVEMYTD GQWNDRNCLY |
| 241 | SRLTICDF |

An amino acid sequence for human pulmonary surfactant associated protein B can be found in the NCBI database as accession number NP 000533 (gi: 4506905). See website at ncbi.nlm.nih.gov. This sequence for human SP-B is provided below as follows (SEQ ID NO:14).

| | |
|---|---|
| 1 | MAESHLLQWL LLLLPTLCGP GTAAWTTSSL ACAQGPEFWG |
| 41 | QSLEQALQCR ALGHCLQEVW GHVGADDLCQ ECEDIVHILN |
| 81 | KMAKEAIFQD TMRKFLEQEC NVLPLKLLMP QCNQVLDDYF |
| 121 | PLVIDYFQNQ IDSNGICMHL GLCKSRQPEP EQEPGMSDPL |
| 161 | PKPLRDPLPD PLLDKLVLPV LPGALQARPG PHTQDLSEQQ |
| 201 | FPIPLPYCWL CRALIKRIQA MIPKGALRVA VAQVCRVVPL |
| 241 | VAGGICQCLA ERYSVILLDT LLGRMLPQLV CRLVLRCSMD |
| 281 | DSAGPRSPTG EWLPRDSECH LCMSVTTQAG NSSEQAIPQA |
| 321 | MLQACVGSWL DREKCKQFVE QHTPQLLTLV PRGWDAHTTC |
| 361 | QALGVCGTMS SPLQCIHSPD L |

In addition, human SP18 (SP-B) surfactant protein may be utilized as described herein. See, e.g., U.S. Pat. Nos. 5,407,914; 5,260,273; and 5,164,369, the disclosures of which are incorporated by reference herein.

An amino acid sequence for human pulmonary surfactant associated protein C can be found in the NCBI database as accession number P11686 (gi: 131425). See website at ncbi.nlm.nih.gov. This sequence for human SP-C is provided below as follows (SEQ ID NO:15).

| | |
|---|---|
| 1 | MDVGSKEVLM ESPPDYSAAP RGRFGIPCCP VHLKRLLIVV |
| 41 | VVVVLIVVVI VGALLMGLHM SQKHTEMVLE MSIGAPEAQQ |
| 81 | RLALSEHLVT TATFSTGSTG LVVYDYQQLL IAYKPAPGTC |
| 121 | CYIMKIAPES IPSLEALNRK VHNFQMECSL QAKPAVPTSK |
| 161 | LGQAEGRDAG SAPSGGDPAF LGMAVNTLCG EVPLYYI |

An amino acid sequence for human pulmonary surfactant associated protein D can be found in the NCBI database as accession number P50404 (gi: 1709879). See website at ncbi.nlm.nih.gov. This sequence for human SP-D is provided below as follows (SEQ ID NO:16).

| | |
|---|---|
| 1 | MLPFLSMLVL LVQPLGNLGA EMKSLSQRSV PNTCTLVMCS |
| 41 | PTENGLPGRD GRDGREGPRG EKGDPGLPGP MGLSGLQGPT |
| 81 | GPVGPKGENG SAGEPGPKGE RGLSGPPGLP GIPGPAGKEG |
| 121 | PSGKQGNIGP QGKPGPKGEA GPKGEVGAPG MQGSTGAKGS |
| 161 | TGPKGERGAP GVQGAPGNAG AAGPAGPAGP QGAPGSRGPP |
| 201 | GLKGDRGVPG DRGIKGESGL PDSAALRQQM EALKGKLQRL |
| 241 | EVAFSRYQKA ALFPDGRSVG DKIFRTADSE KPFEDAQEMC |
| 281 | KQAGGQLASP RSATENAAIQ QLITAHNKAA FLSMTDVGTE |
| 321 | GKFTYPTGEP LVYSNWAPGE PNNNGGAENC VEIFTNGQWN |
| 361 | DKACGEQRLV ICEF |

A related peptide is the WMAP-10 peptide (Marion Merrell Dow Research Institute) having the sequence succinyl-Leu-Leu-Glu-Lys-Leu-Leu-Gln-Trp-Lys-amide (SEQ ID NO:17). Alternative peptides are polymers of lysine, arginine or histidine that induce a lowering of surface tension in admixtures of phospholipids as described herein.

In still another embodiment, a polypeptide of this invention has amino acid residue sequence that has a composite hydrophobicity of less than zero, preferably less than or equal to −1, more preferably less than or equal to −2. Determination of the composite hydrophobicity value for a peptide is known in the art, see, U.S. Pat. No. 6,013,619, the disclosure of which is incorporated herein by reference. These hydrophobic polypeptides perform the function of the hydrophobic region of SP18. Thus, in one preferred embodiment, the amino acid sequence mimics the pattern of charged and uncharged, or hydrophobic and hydrophilic, residues of SP18.

It should be understood, however, that polypeptides and other surfactant molecules of the present invention are not limited to molecules having sequences like that of native SP-B (SP18). On the contrary, some of the most preferred surfactant molecules of the present invention have little resemblance to SP18 with respect to a specific amino acid residue sequence, except that they have similar surfactant activity and alternating charged/uncharged (or hydrophobic/hydrophilic) residue sequences.

One disclosed embodiment of the present invention comprises a peptide-containing preparation, the 21-residue peptide being a mimic of human SP-B consisting of repeated units of four hydrophobic leucine (L) residues, bounded by basic polar lysine (K) residues. This exemplary peptide, which is abbreviated herein as “KL₄,” has the following amino acid residue sequence:

| | |
|---|---|
| KLLLLKLLLLKLLLLKLLLLK. | (SEQ ID NO 1) |

In one embodiment, KL₄ is combined with phospholipids dipalmitoyl phosphatidylcholine and palmitoyl-oleoylphosphatidyl glycerol (3:1) and palmitic acid, the phospholipid-peptide aqueous dispersion has been named “KL₄-Surfactant,” and it is generally referred to herein in that manner. The KL₄-surfactant is being marketed under the name Model surfactant mixture. The efficacy of KL₄-Surfactant in various experimental and clinical studies has been previously reported, see, e.g., Cochrane et al, Science, 254:566-568 (1991); Vincent et al., Biochemistry, 30:8395-8401 (1991); Cochrane et al., Am J Resp & Crit Care Med, 152:404-410 (1996); and Revak et al., Ped. Res., 39:715-724 (1996).

In various embodiments of the present invention, the polypeptide: phospholipid weight ratio is in the range of about 1:5 to about 1:10,000, preferably about 1:7 to about 1:5,000, more preferably about 1:10 to about 1:1,000, and most preferably about 1:15 to about 1:100. In a particular preferred embodiment, the polypeptide:phospholipid weight ratio is about 1:37.

Synthetic polypeptides suitable for preparing the carrier surfactant composition in accordance with the present invention can be synthesized from amino acids by techniques that are known to those skilled in the polypeptide art. An excellent summary of the many techniques available may be found in J. M. Steward and J. D. Young, SOLID PHASE PEPTIDE SYNTHESIS, W.H. Freeman Co., San Francisco, 1969, and J. Meienhofer, HORMONAL PROTEINS AND PEPTIDES, Vol. 2, p. 46, Academic Press (New York), 1983 for solid phase peptide synthesis, and E. Schroder and K. Kubke, THE PEPTIDES, Vol. 1, Academic Press (New York), 1965 for classical solution synthesis.

In general, these methods comprise the sequential addition of one or more amino acid residues or suitably protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group (e.g., lysine).

Example 1 illustrates a solid phase synthesis of the surfactant peptide. Briefly, a protected or derivatized amino acid is attached to an inert solid support through its unprotected carboxyl or amino group. The protecting group of the amino or carboxyl group is then selectively removed and the next amino acid in the sequence having the complementary (amino or carboxyl) group suitably protected is admixed and reacted under conditions suitable for forming the amide linkage with the residue already attached to the solid support. The protecting group of the amino or carboxyl group is then removed from this newly added amino acid residue, and the next amino acid (suitably protected) is then added, and so forth. After all the desired amino acids have been linked in the proper sequence, any remaining terminal and side group protecting groups (and any solid support) are removed sequentially or concurrently, to afford the final polypeptide. That polypeptide is then washed by dissolving in a lower aliphatic alcohol, and dried. The dried surfactant polypeptide can be further purified by known techniques, if desired.

The surfactant proteins and polypeptides of the present invention may also be produced by recombinant DNA technology. The procedure of deriving protein molecules from the plant or animal hosts are generally known in the art. See, Jobe et al., Am. Rev. Resp. Dis., 136:1032 (1987); Glasser et al., J. Biol. Chem., 263:10326, (1988). Generally, a gene sequence encoding the proteins or polypeptides under the control of a suitable promoter and/or signal peptide is inserted into a plasmid or vector for transfection of a host cells. The expressed proteins/polypeptide may be isolated from the cell culture.

While it is appreciated that many useful polypeptides disclosed herein, e.g., the KL₄ polypeptide (SEQ ID NO:1), comprise naturally-occurring amino acids in the “L” form that are joined via peptide linkages, it should also be understood that molecules including amino acid side chain analogs, non-amide linkages (e.g., differing backbones) may also display a significant surfactant activity and may possess other advantages, as well. For example, if it is desirable to construct a molecule (e.g., for use in a surfactant composition) that is not readily degraded, one may wish to synthesize a polypeptide molecule comprising a series of D-amino acids. Molecules comprising a series of amino acids linked via a “retro” backbone, i.e., a molecule that has internal amide bonds constructed in the reverse direction of carboxyl terminus to amino terminus, are also more difficult to degrade and may thus be useful in various applications, as described herein. For example, the following illustrates an exemplary molecule with a “retro” bond in the backbone:

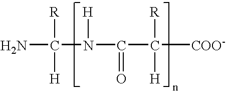

In another variation, one may wish to construct a molecule that adopts a more “rigid” conformation; one means of accomplishing this would be to add methyl or other groups to the α-carbon atom of the amino acids.

As noted above, other groups besides a CH₃ group may be added to the alpha carbon atom, that is, surfactant molecules of the present invention are not limited to those incorporating a CH₃ at the a carbon alone. For example, any of the side chains and molecules described above may be substituted for the indicated CH₃ group at an a carbon component.

As used herein, the terms “analogs” and “derivatives” of polypeptides and amino acid residues are intended to encompass metabolites and catabolites of amino acids, as well as molecules that include linkages, backbones, side-chains or side-groups that differ from those ordinarily found in what are termed “naturally-occurring” L-form amino acids. (The terms “analog” and “derivative” may also conveniently be used interchangeably herein.) Thus, D-amino acids, molecules that mimic amino acids and amino acids with “designed” side chains (i.e., that can substitute for one or more amino acids in a molecule having surfactant activity) are also encompassed by the terms “analogs” and “derivatives” herein.

A wide assortment of useful surfactant molecules, including amino acids having one or more extended or substituted R or R′ groups, is also contemplated by the present invention. Again, one of skill in the art should appreciate from the disclosures that one may make a variety of modifications to individual amino acids, to the linkages, and/or to the chain itself, which modifications will produce molecules falling within the scope of the present invention, as long as the resulting molecule possesses surfactant activity as described herein.

The composition can include other ingredients. For example, the surfactant mixture of the invention can includes (i) 50-95 dry weight percent phospholipid, (ii) 2-25 dry weight percent of a spreading agent effective to promote incorporation of the phospholipid into the surface lining layer of the lung, and (iii) 0.1 to 10 dry weight percent of lung-surfactant polypeptide. As indicated above, the components may be mixed in dry, solution, or particle-suspension form, and may be preformulated, prior to addition of the therapeutic agent, or may be formulated together with the agent.

Phospholipids useful in the compositions of the invention include native and/or synthetic phospholipids. Phospholipids that can be used include phosphatidylcholines, phospatidylglycerols, phosphatidylserines, phosphatidic acids, and phosphatidylethanolamines. Exemplary phospholipids also include phosphatidylcholines, such as dipalmitoyl phosphatidylcholine (DPPC), dilauryl phosphatidylcholine (DLPC) C12:0, dimyristoyl phosphatidylcholine (DMPC) C14:0, distearoyl phosphatidylcholine (DSPC), diphytanoyl phosphatidylcholine, nonadecanoyl phosphatidylcholine, arachidoyl phosphatidylcholine, dioleoyl phosphatidylcholine (DOPC) (C18:1), dipalmitoleoyl phosphatidylcholine (C16:1), linoleoyl phosphatidylcholine (C18:2)), dipalmitoyl phosphatidylethanolamine, dioleoylphosphatidylethanolamine (DOPE), dioleoyl phosphatidylglycerol (DOPG), palmitoyloleoyl phosphatidylglycerol (POPG), distearoylphosphatidylserine (DSPS) soybean lecithin, egg yolk lecithin, sphingomyelin, phosphatidylserines, phosphatidylglycerols, phosphatidyl mositols, diphosphatidyl glycerol, phosphatidylethanolamine, and phosphatidic acids.

In particular, 1,2-diacyl-sn-glycero-3-[phospho-rac-(1-glycerol)], 1,2-diacyl-sn-glycero-3-[phospho-L-serine], 1,2 diacyl-sn-glycero-3-phosphocholine, 1,2-diacyl-sn-glycero-3-phosphate, 1,2-diacyl-sn-glycero-3-phosphoethanolamine where the diacyl groups may be symmetrical, asymmetrical and contain either saturated or unsaturated fatty acids of various types ranging from 3 to 28 carbons in chain length and with up to 6 unsaturated bonds.

One preferred phospholipid is DPPC. DPPC is the principal phospholipid in all mammalian species examined to date. DPPC is synthesized by epithelial cells of the airspaces (the type 2 pneumocyte of the alveoli and an as yet unidentified cell of the airways). DPPC is secreted into a cellular lining layer and spreads out to form a monomolecular film over the alveoli. The DPPC film at the air-cellular lining interface has certain unique properties that explain its normal function: (1) the film, which spreads to cover all surfaces, achieves extremely low surface tension upon compression, e.g., during exhalation, thereby reducing the net force that favors liquid movement into the airspace; (2) as airway or alveolar size falls, surface tension falls proportionately, thereby establishing a pressure equilibration among structures to prevent collapse; (3) because of its amphoteric structure, the film can form loose chemical associations with both hydrophobic and hydrophilic moieties and because of its high compressibility these associations can be broken upon film compression, thereby freeing the moiety from the interface; and (4) these loose chemical associations can be modified by the addition of other compounds found in the surfactant system (PG, for example) that can alter the charge distribution on the film, thereby altering the rate at which the moiety (as mentioned in (3) above) is released from the film.

In various embodiments of the invention, the lipid component is DPPC that comprises about 50 to about 90 weight percent of the surfactant carrier composition. In another embodiment of the invention, DPPC comprises about 50 to 75 weight percent of the surfactant composition with the remainder comprising unsaturated phosphatidylcholine, phosphatidylglycerol (PG), triacylglycerols, palmitic acid, spingomyelin or admixtures thereof. In yet another embodiment of the invention, the lipid component is an admixture of DPPC and POPG in a weight ratio of about between 4:1 and 2:1. In one preferred embodiment, the lipid component is an admixture of DPPC and palmitoyl-oleoyl phosphatidylglycerol (POPG) in a weight ratio of about 3:1.

DPPC and the above-described lipids and phospholipids can be obtained commercially, or prepared according to published methods that are generally known in the art. The phospholipid component of the mixture includes one or more phospholipids, such as phosphatidylcholine (PC), phosphatidyl ethanolamine (PE), phosphatidylinositol (PI), phosphatidyl glycerol (PG), phosphatidic acid (PA), phosphatidyl serine (PS), and sphingomyelin (SM). The fatty acyl chains in the phospholipids are preferably at least about 7 carbon atoms in length, typically 12-20 carbons in length, and may be entirely saturated or partially unsaturated. It is known that phospholipids, such as DPPC, are absorbed relatively slowly to the air-cell lining interface when administered alone and, once adsorbed, spread slowly.

The phospholipid(s) make up 50-95 dry weight percent of the surfactant mixture, and preferably between 80-90 percent by dry weight of the mixture.

While do not wishing to be limited to a specific mechanism, the spreading agent is believed to promote transition of surfactant-mixture lipids from particle form to monolayer form, leading to spreading on and distribution along and within the lung surface. Thus, for example, if the surfactant formulation is delivered to the lung in liposomal form, the spreading agent is effective in promoting transition of the liposomal phospholipids from liposomal bilayer to a planar monolayer form at the lung surface. Similarly, if the surfactant formulation is delivered to the lung as amorphous or crystalline lipid particles, the spreading agent is effective in promoting transition of the surfactant-mixture phospholipids to a planar monolayer form at the lung surface.

Exemplary spreading agents include but are not limited to non-phospholipid lipids that are compatible with lipid bilayer or lipid monolayer formation, but which alone are not able to support lipid-bilayer formation. Exemplary spreading agents include lysophospholipids; fatty acids, fatty esters, and fatty alcohols, and other single-long-chain fatty acyl compounds. Preferred spreading agents include fatty acids and fatty alcohols having alkyl chain lengths of at least about 12 carbon atoms, preferably between 15-20 carbon atoms in chain length. One preferred spreading agent is palmitic acid; another is cetyl alcohol.

The spreading agent makes up about 2 to about 25 dry weight percent of the surfactant mixture, or about 10 to about 15 dry weight percent of the mixture. One exemplary mixture, also containing DPPC:POPG (3:1) at 84.5% dry weight, contains 12.75 dry weight percent palmitic acid.

The spreading agents used in the present invention may be purchased from commercial suppliers. For example, palmitic acid (PA) may be obtained from Avanti Polar Lipids, Inc. (Birmingham, Ala.). The spreading agents may also be prepared according to published methods that are generally known in the art.

In some embodiments, the composition can include Tyloxapol as a spreading agent, which can be purchased under several trade names from various companies such as Sterling-Winthrop, and Rohm and Haas. Tyloxapol is a polymer of 4-(1,1,3,3-tetramethylbutyl)phenol) with formaldehyde and oxirane. Tyloxapol has been used in human pharmacologic formulations for over 30 years (Tainter M L et al. New England Journal of Medicine (1955) 253:764-767). Tyloxapol is relatively nontoxic and does not hemolyze red blood cells in a thousand times the concentrations at which other detergents are hemolytic (Glassman HN. Science (1950) 111:688-689).

Other compounds can be included in the compositions of the invention, including those compatible with or suitable for treating asthmatic conditions. Agents that can be co-administered include anti-allergenic agents, anti-inflammatory agents, anti-microbials including anti-bacterials, anti-fungals, and anti-virals, antibiotics, immunomodulators, hematopoietics, leukotriene modifiers, xanthines, sympathomimetic amines, mucolytics, corticosteroids, anti-histamines, and vitamins. Other examples include bronchodilators, such as albuterol, levalbuterol (e.g., Xopenex®), terbutaline, salmeterol, formoterol, and pharmacologically acceptable salts thereof, anticholinergics, such as ipratropium bromide, the so-called “mast cell stabilizers,” such as cromolyn sodium and nedocromil, corticosteroids, such as flunisolide, fluticasone, beclomethasone, budesonide, triamcinolone, and salts thereof, interferons such as INF-alpha, beta and gamma, mucolytics, such as N-acetylcysteine and guaifenesin, leukotriene antagonists, such as zafirlukast and montelukast, phosphodiesterase IV inhibitors, antibiotics, such as amikacin, gentamycin, colistin, protegrins, defensins and tobramycin, antiviral agents, such as ribavirin, RSV monoclonal antibody, VP14637, antitubercular agents, such as isoniazid, rifampin, and ethambutol, and antifungal agents, such as amphoterecin B.

Treatment Methods

The invention provides compositions and methods for treating asthma, including, for example, acute inflammatory asthma, allergic asthma, iatrogenic asthma and related asthmatic conditions.

Asthma is a reversible obstructive pulmonary disorder (ROPD) characterized by increased responsiveness of the airway, resulting in airway obstruction. Airway obstruction is defmed as an increased resistance to air flow during forced expiration. In asthma, airway obstruction typically results from bronchospasm, bronchial wall edema and bronchiolar collapse. The underlying mechanisms causing asthma are unknown, but inherited or acquired imbalance of adrenergic and cholinergic control of airway diameter has been implicated. Asthmatics manifesting such imbalance have hyperactive bronchi and, even without symptoms, bronchoconstriction may be present. In addition, dysfunction of surfactant lining bronchial airways has been implicated in the induction of airway obstruction, leading to alveolar hyper-expansion. Overt asthma attacks may occur when such individuals are subjected to various stresses, such as viral respiratory infection, exercise, emotional upset, nonspecific factors (e.g., changes in barometric pressure or temperature), inhalation of cold air or irritants (e.g., gasoline fumes, fresh paint and noxious odors, or cigarette smoke), exposure to specific allergens, and ingestion of aspirin or sulfites in sensitive individuals. Those whose asthma is precipitated by allergens (most commonly airborne pollens and molds, house dust, animal danders) and whose symptoms are IgE-mediated are said to have allergic or “extrinsic” asthma. They account for about 10 to 20% of adult asthmatics; in another 30 to 50%, symptomatic episodes seem to be triggered by non-allergenic factors (e.g., infection, irritants, emotional factors), and these patients are said to have non-allergic or “intrinsic” asthma. In many persons, both allergenic and non-allergenic factors are significant.

The treatment methods of the invention employ a surfactant mixture having at least one of the lung surfactant polypeptides of the invention. The formulation can be a liquid or dry formulation. The formulation can be formulated for inhalation, for example, as an aerosol or for delivery by a nebulizer. Alternatively, the formulation can be formulated for liquid bolus administration. The amount of formulation administered to a patient is typically about 1-100 mg/dose, 5-20 mg/dose, e.g., 10 mg/dose, and the amount of active agent in the dose is a therapeutically effective amount, e.g., about 0.01 mg to 50 mg drug or about 0.01 mg to 5 mg drug. Adjustments to the dose, to optimize therapeutic effectiveness, and minimize side effects, can be determined according to known procedures that may involve animal models of asthma, pulmonary inflammation and/or clinical studies on human patients with asthmatic conditions.

Thus, in one embodiment, the invention contemplates a method for treating asthma in a mammal comprising administering to the mammal a therapeutically effective amount of a composition comprising a lung surfactant polypeptide of the invention.

As noted above, the invention is advantageously used for treating a variety of asthmatic conditions, including those in which no inflammatory component is involved. Asthma and related broncho-constriction conditions may be also treated by administering a surfactant formulation containing bronchodilators, such as albuterol, terbutaline, salmeterol, formoterol, and pharmacologically acceptable salts thereof. The present compositions can therefore also include other usefil agents, such as the bronchodilators described above, corticosteroids, anti-asthma medications, leukotriene modifiers, antibiotics, pain medicaments, or polypeptides, such as cytokines, and peptide hormones.

Compositions

The surfactant mixtures of the invention may be formulated into a variety of acceptable compositions. Such pharmaceutical compositions can be administered to a mammalian host, such as a human patient, in a variety of forms adapted to the chosen route of administration, i.e., by pulmonary or inhalation routes.

In cases where polypeptide surfactants or other compounds, are sufficiently basic or acidic to form stable nontoxic acid or base salts, administration of such compounds as salts, together with the phospholipids, may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts. Pharmaceutically acceptable salts are obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids also are made.

Pharmaceutically acceptable salts of polypeptides include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Generally, the concentration of the lung surfactant polypeptides of the present invention in a composition will be from about 0.01 to 10 weight-percentage of the phospholipids.

In general, however, a suitable dose will be in the range of from about 0.1 to about 300 mg phospholipid per kilogram, or from about 0.1 to about 200 mg phospholipid per kilogram, e.g., from about 1.0 to about 150 mg phospholipid per kilogram of body weight per day, such as 1 to about 50 mg phospholipid per kilogram of body weight per day, or in the range of 3 to 90 mg phospholipid per kilogram of body weight per day or in the range of 5 to 60 mg phospholipid per kilogram of body weight per day, and containing the lung surfactant polypeptide in the percentages specified above.

Ideally, the lung surfactant polypeptides and phospholipids should be administered to achieve optimal treatment of asthmatic conditions. The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator.

For example, in some embodiments, an aerosolized surfactant mixture containing 1-25 mg phospholipid and 0.01 to 10 weight percentage lung surfactant polypeptide can be deposited in the lungs over a 2 to 30 minute period. Treatments may be repeated to increase air flow as needed in the bronchi.

The surfactant polypeptides and phospholipids contemplated for use in the present invention can be delivered directly to the site of interest (the lung) to provide immediate relief of the symptoms of asthma or pulmonary inflammation. Such delivery can be by bronchoalveolar lavage, intratracheal administration, inhalation or aerosol administration.

Therapeutic compositions of the present invention may contain a physiologically tolerable carrier together with surfactant mixtures, as described herein, dissolved or dispersed therein as an active ingredient. In a preferred embodiment, the therapeutic composition is not immunogenic when administered to a mammal or human patient for therapeutic purposes.

The preparation of a pharmacological composition that contains active ingredients dissolved or dispersed therein is well understood in the art and need not be limited based on formulation. The active ingredients (lung surfactant polypeptides and phospholipids) can be mixed with excipients that are pharmaceutically acceptable and compatible with the active ingredient and in amounts suitable for use in the therapeutic methods described herein. Suitable excipients are, for example, water, saline, buffered solutions or the like and combinations thereof. In addition, if desired, the composition can contain minor amounts of auxiliary substances such as wetting or emulsifying agents, pH buffering agents and the like which enhance the effectiveness of the active ingredients.

Exemplary of liquid carriers are sterile aqueous solutions that contain no materials in addition to the active ingredients and water, or contain a buffer such as sodium phosphate or tromethamine buffers at physiological pH value, physiological saline or both, such as phosphate-buffered saline or sodium chloride fortified tromethamine buffer. Still further, aqueous carriers can contain more than one buffer salt, as well as salts such as sodium and potassium chlorides, dextrose, polyethylene glycol and other solutes

In some embodiments, the liquid carrier is a Tham buffered system, which can be prepared essentially as follows. 0.37 ml of Tham solution (tromethamine injection, NDC 0074-1593-04, Abbott Laboratories, North Chicago, Ill.), with the pH adjusted using acetic acid (AR Select, ACS, Mallinckrodt, Paris, Ky.) to a pH of 7.2±0.5, is admixed with 0.33 ml saline (0.9% sodium chloride injection, USP, Abbott Laboratories) and 0.30 ml water (sterile water for injection, USP, Abbott Laboratories). The solution can be sterilized by sterile-filtration.

In one embodiment, a “surfactant mixture” is prepared that refers to a mixture of phospholipid, spreading agent, and lung-surfactant protein. The surfactant mixture may be processing into a lipid-body formulation such as a liposome suspension. The surfactant formulation may constitute well-defined lipid bodies, for example, liposomes that incorporate the lung surfactant polypeptides, lipid-crystal or amorphous lipid bodies containing both surfactant mixture and active agent components, a solution of the components in an organic solvent or organic/aqueous co-solvent, or a dispersion in which some of the some are in lipid-body form, and other components in solute form.

For aerosol administration, the only composition and structural requirements of the surfactant formulation is that be it can be converted or processed into a suitable aerosol-particle form containing all of the above lipid and lung surfactant polypeptide components.

Considering now the various processing steps contemplated by the invention, the surfactant formulation, preferably as a aqueous suspension of lipid bodies, is lyophilized to form a dry mass that is then comminuted, e.g., by grinding, to form a composition containing dry-powder particles having a mass median aerodynamic diameter in the 1-5 μm size range. The dry-powder particles are then stored and employed in a suitable aerosolization device to produce a dry-particle aerosol suitable for inhalation treatment or for suspension in a suitable solvent, for aerosolization as a particle suspension.

In another embodiment, the invention contemplates processing a liquid surfactant formation by means of a user-controlled nebulizer or aerosolizer, to generate an aqueous-droplet aerosol containing the surfactant formulation in lipid-body form. The surfactant formulation components of this embodiment can be present in ordered, crystalline, or amorphous lipid particles suspended in the aerosol droplets.

In still another embodiment, the surfactant formulation is processed by spray drying to produce spray-dried particles having the desired mass median aerodynamic diameter in the 1-5 μm size. The spray dried particles may then be stored and employed by the user in an aerosolization device, as above, for inhalation therapy. As indicated, the powdered particles can be delivered as a dry-powder aerosol, or the particles can be suspended in an aqueous medium for aerosolization in aqueous droplet form. Alternatively, a suitable surfactant formulation in liquid form, e.g., a formulation solution or suspension contained in a volatile biocompatible fluid, may be formed in an aerosolization process in which the particles formed are immediately inhaled for therapeutic delivery of the active agent.

As noted above, the formulation of the invention can be prepared as a solution formulation or as a particulate formulation. The lipid components or the therapeutic agent, or both can also be incorporated into liposomal, crystalline, or amorphous lipid bodies suspended in an aqueous, organic, or mixed solvent.

A dispersion of liposomes (lipid vesicles) may be made by a variety of techniques, such as those detailed in Szoka, F. Jr., et al., Ann. Rev. Biophys. Bioeng., 9:467-508, 1980. Liposomal-like surfactant compositions of the present invention are generally sterile liposome suspensions. These liposomes may be multiple compartment or multilamellar vesicles, single compartment vesicles, macrovesicles or other colloidal forms. The multilamellar vesicles are generally the most common. Multilamellar vesicles (MLVs) can be formed by simple lipid-film hydration techniques, preferably under sterile condition.

One method for producing a liposomal-like surfactant composition involves dissolving the surfactant polypeptide in an organic solvent together with the selected phospholipids, and then combining the resulting solution with an aqueous buffer solution. The resulting dispersion is then dialyzed to remove the organic solvent. Alternatively, the organic solvent can be removed by evaporation and/or exposure to a vacuum. The dried lipid/polypeptide mixture thus produced is rehydrated in an aqueous buffer system to produce the liposomes (Olson, F., et al., Biochim. Biophys. Acta, 557:9-23, 1979).

Suitable buffers include Tris buffers, a Tham buffer system and the like used. Tham is a buffering agent also known as Tris, tromethamine, and tris(hydroxymethyl)aminomethane. In various preferred embodiments, the compositions have a pH range of about 6.5-8.0.

Liposomes may be sized by extruding the aqueous dispersion of liposomes through a series of polycarbonate membranes having a selected uniform pore size. The pore size of the membrane corresponds roughly to the largest sizes of liposomes produced by extrusion through that membrane, particularly where the preparation is extruded two or more times through the same-sized membrane. The liposomes so produced can be in the range of 0.03 to 5 micron. Homogenization and sonication methods are also useful for down-sizing liposomes to average sizes of 100 nm or less (Martin, F. J., In: SPECIALIZED DRUG DELIVERY SYSTEMS-MANUFACTURING AND PRODUCTION TECHNOLOGY, P. Tyle, ed., Marcel Dekker, New York, pp. 267-316, 1990).

If it is desired to incorporate the therapeutic agent into the liposomes prior to liposome formation, this may be done by standard techniques. For example, if the liposomes are formed by lipid hydration, a hydrophobic drug can be included in the lipid mixture to be hydrated and a hydrophilic drug can be incorporated into the hydration solution. High encapsulation efficiency of hydrophilic compounds, e.g., proteins, can be achieved by employing the reverse evaporation phase method, in which drug-containing aqueous medium is added to partially evaporated lipid structures.

Another method for achieving high encapsulation efficiencies for hydrophilic drugs is by solvent injection, where a lipid solution in a volatile organic solvent, e.g., ether, is injected into an aqueous solution of drug. With continued injection of the lipid solution to high lipid concentration, very high encapsulation rates, e.g., 50% of greater, may be achieved.

The solvent injection involves addition of an aqueous solution of hydrophilic drug or organic solution of hydrophobic drug to a co-solvent dispersion of lipids (containing the surfactant mixture components), concomitant with or followed by aqueous dilution and evaporation of the organic solvent, to form a bulk formulation of lipid particles, e.g., liposomes, with incorporated or encapsulated drug.

Alternatively, an additional active agent may be added to the preformed liposomes. In this case, the surfactant polypeptide-lipid mixture comprises pre-formed liposomes. If the compound is a hydrophobic compound, the compound may be simply contacted with the liposomes, for uptake into the bilayer membrane by partitioning out of aqueous phase medium. For ionizable, hydrophilic and amphipathic compounds, high internal encapsulation into preformed liposomes can be achieved by loading the drug against a pH or other ion gradient, e.g., an ammonium gradient, according to available methods.

The formulation of liposomes may be stored as a lipid dispersion, for aerosolization in aqueous-droplet form, or the liposome formulation may be lyophilized, powdered, and administered as a dry-powder aerosol. Alternatively, a liposome dispersion may spray-dried, forming dried lipid particles in powder form, for administration as a powdered aerosol.

Freeze drying (lyophilization) is one standard method for producing a dry powder from a solution or a suspension. See, for example, Freide, M., et al., Anal. Biochem., 211 (1):117-122, 1993; Sarbolouki, M. N. and T. Toliat, PDA J. Pharm. Sci. Technol., 52 (1):23-27, 1998). Following lyophilization, the dried surfactant formulation is comminuted, e.g., by grinding or other conventional means, to form desired size particles.

Recently, techniques that make use of the supercritical properties of liquefied gases have been employed in the generation of microparticles and powders containing therapeutic proteins (Niven, R. W., In: MODULATED DRUG THERAPY WITH INHALATION AEROSOLS: REVISITED, A. J. Hickey, ed., Marcel Dekker, New York). Particles with preferred crystal habits and characteristics suitable for inhalation purposes can be prepared by these methods. Exemplary supercritical fluid processing techniques include: rapid expansion of supercritical fluids (RESS), the use of gas-antisolvent (GAS) precipitation to prepare particles, and the solution-enhanced dispersion of supercritical fluids (SEDS) (see, U.S. Pat. Nos. 5,301,644; 5,707,634; 5,770,559; 5,981,474; 5,833,891; 5,874,029, and 6,063,138).

Spray drying may also be used advantageously for producing dried lipid particles of desired sizes. (See, Master, K., SPRAY DRYING HANDBOOK, 5^{th} edition, J. Wiley & Sons, New York, 1991; Maa, Y. F. et al., Pharm. Res., 15 (5):768-775, 1998; Maa, Y. F., Pharm. Dev. Technol., 2 (3):213-223, 1997). Various spray-drying methods have been described in the patent literature, See, for example, U.S. Pat. Nos. 6,174,496; 5,976,574; 5,985,284; 6,001,336; 6,015,256; 5,993,805; 6,223,455; 6,284,282; and 6,051,257.

One spray-drying device that can be used is a cyclone drier that has a drying tank. The liquid mixture is fed into the drying tank and warm gas, e.g., air or nitrogen, or another inert gas is forced into the top of the tank. The feed liquid is broken up as it enters the tank, and dried by the warm gas as it is carried toward the bottom of the tank, and from there, to a collection unit. According to known processing parameters, the solvent, rate of injection, and rate of warm-gas flow can be adjusted to produce the desired-size dried particles. In this case, particles having a mean hydrodynamic diameter, for example, in the 1-5 μm range can be used. In the procedure, the drying temperature is at least about 37 degrees C., and preferably higher than 40 degrees C. and may be well over 100 degrees C. The temperature within the collection chamber is substantially lower than that of the heated air.

A hydrophobic or hydrophilic drug can be added to a suitable co-solvent solution that also contains the surfactant-mixture components. The resulting mixture is spray dried to produce the desired-sized dry particles in a bulk powder formulation. These particles can then be packaged and stored, preferably under dry conditions, until used in an aerosolizer for administering the dried particles to the lungs.

Both amorphous particles having a variety of morphologies and crystalline powder particles with well-defined crystalline shapes can be utilized so long as the particle size is not too large. Both types of particles are suitable for the invention, although it is preferable that the particles, once formed, be maintained in the initial state, since transition between the two states can affect the chemical and physical stability of the active pharmaceutical ingredients and can directly influence the ability of powders to be dispersed and deaggregated from inhaler devices. These changes may also influence the pharmacokinetic properties of the particles. In general, the factors that influence the tendency of amorphous powders to undergo a transition to crystalline form include moisture, the presence of hydrophilic agents, impurities, temperature, and time. Factors that may reduce the tendency of amorphous particles to undergo transition to a crystalline state are the presence of protein and polymers, and hydrophobic materials. Of these several factors that affect transition, the most important are temperature and moisture, highlighting the need to store the particles, prior to aerosolization, in a dry state under moderate storage temperatures.

Regardless of the method of forming suspended or dried particles, the particles are formed under conditions that give a desired MMAD in the range 1-5 microns. Where the particles are intended to carry the lung surfactant polypeptide(s) deep into the lungs, such as for treatment of an asthmatic lung condition affecting tissues deep in the lungs, the particles are preferably predominantly in the 1-3 or 1-2 micron MMAD size range. Where delivery of the lung surfactant polypeptide is targeted to the airways, larger particle sizes, e.g., in the 3-5 MMAD size range, may be more appropriate.

Where the formulation is an aqueous suspension of liposomes or other lipid particles, a variety of commercial nebulizers may be used to produce the desired aerosol particles. Typically, the nebulizing operation is carried out at a pressure of about 10-50 psig, and the aqueous particles formed are typically in the range of about 2-6 microns. The device may be controlled to produce a measured quantity of aerosolized liposomes or lipid-based particles, according to known operational variables.

Another device suitable for aerosolizing an aqueous dispersion of liposomes, and preferably a relatively dilute dispersion containing less than about 25%-30% encapsulated aqueous volume, uses ultrasonic energy to break up a carrier fluid into a fine mist of aqueous particles. The ultrasonic nebulizer device has been found to produce a liposome aerosol mist whose particle sizes are about the same as those formed by a compressed air nebulizer, i.e., between about 2-6 microns.

For aerosolizing a concentrated liposome dispersion of the type used for delivery of a water-soluble, liposome-permeable drug, the dispersion is first mixed with a carrier solvent, to form a diluted dispersion that can be aerosolized. The carrier solvent may be an aqueous medium, in which case the dispersion is diluted or adapted to a form suitable for spraying, such as by a pneumatic or ultrasonic nebulizer. The amount of additive added is sufficient to render the dispersion suitable for spraying and, for example, contains less than about 30% total encapsulated volume. Assuming the dispersion has an initial encapsulated volume of 70-75% of the total dispersion volume, it can be appreciated that a given volume of the dispersion must be diluted with at least one or two volumes of diluent.

Alternatively, the surfactant components may be dissolved or suspended in a suitable volatile, biocompatible solvent, such as given below, and sprayed from a suitable aerosolizer device under conditions that (i) lead to initial formation of spray dried particles and (ii) inhalation of the just-formed particles into the lungs.

This section describes various self-contained delivery devices designed for producing an airborne suspension of the dried lipid particles. As defined herein “self-contained” means that the particle aerosol is produced in a self-contained device that it propelled by a pressure differential created either by release of a pressurized fluorochlorocarbon propellant or by a stream of air drawn through or created in the device by the user. It will be appreciated that conventional powered aerosolizers for dry powders are also suitable.

Lipid particle/propellant suspensions can also be utilized in the invention with a conventional pressurized propellant spray device for delivering a metered amount of dried lipid particles that are suspended in the propellant. Because the system requires long-term suspension of lipid particles, e.g., liposomes, in a suitable propellant, the lipid particles and propellant components of the suspension must be selected for stability on storage.

Several fluorochlorocarbon propellant solvents have been used or proposed for self-contained inhalation devices. Representative solvents includes “Freon 11” (CCl₃F), “Freon 12” (CCl₂ F₂), “Freon 22” (CHClF₂), “Freon 113” (CCl₂FCClF₂), as well as others. To form lipid-particle/propellant suspension, the dried lipid particles are added to the selected propellant or propellant mixture, to a final lipid particle concentration of about 1 to 30, and preferably between about 10-25 percent by weight of the total propellant. Where the drug is a water-soluble compound that remains encapsulated in the dried lipid particles of the propellant suspension, the final concentration of lipid particles in the propellant is adjusted to yield a selected metered dose of the drug, in a given aerosol suspension volume. Thus, for example, if liposomes are formulated to contain 0.05 mg lung surfactant polypeptide per mg dried liposome preparation, and the selected dose of drug to be administered is 1 mg, the suspension is formulated to contain 20 mg of dried liposomes per aerosol dose.

If a lipid-soluble drug is to be included in the formulation, i.e., one that is readily soluble in the propellant solvent, two formulation approaches are possible. In the first, the drug is initially included in the lipids used in forming the dried lipid particles, and these are then added to the propellant in an amount that gives a selected concentration of drug/volume of propellant, as above. Alternatively, the drug may be added initially to the solvent, at a selected drug concentration. The lipid particles in this formulation are “empty” dried particles that will act as a lipid reservoir for the drug during aerosol formation and solvent evaporation. The final concentration of empty lipid particles is adjusted to give a convenient total lipid dose that is suitable for holding the metered amount of drug.

Lipid-particle entrainment in a propellant can also be utilized in the invention. In this system, dried lipid particles containing a metered-dose quantity of lung surfactant polypeptides are prepackaged in dehydrated form in a delivery packet. The packet is used with a propellant spray device, to eject the liposome contents of the packet in an airborne suspension of liposome particles.

Lipid-particle entrainment in air can also be utilized in the invention. A third type of delivery system uses an air stream produced by user inhalation to entrain dried lipid particles and draw these into the user's respiratory tract. In operation, a packet is placed on the nozzle, preferably in a manner that ruptures the seal at the “inner” end of the packet, as above, and the other end of packet is unsealed. The user now places his or her lips about the mouthpieces and inhales forcefully, to draw air rapidly into and through a pipe in the inhaler. The air drawn into the pipe becomes concentrated at the nozzle, creating a high-velocity air stream that carries lipid particles out of the packet and into the convection region. The air stream and entrained liposomes impinge on the paddle, causing it to rotate and set up a convection current. The lipid particles are thus distributed more evenly, and over a broader cross section, just prior to being drawn into the user's respiratory tract by inhalation.

Alternatively, the lipid particles could be retained within a device that provides the force required to disperse and aerosolize the powder independent of the inhaled breath of the patient. The timing of dosing within the inhalation maneuver may also be controlled by sensors incorporated within the delivery system.

In other embodiments, the compositions can be administered by liquid bolus administration. For example, a tracheal tube may be positioned to deliver drops of the composition to pulmonary tissues. In some embodiments, bolus administration can be to one portion of the lung and not to another, or different portions of the lung can be treated by bolus drip administration at different times.

In still other embodiments, the compositions can be administered by pulmonary lavage. Procedures for performing pulmonary lavage are available in the art. See, e.g., U.S. Pat. No. 6,013,619, which is incorporated herein by reference. For example, pulmonary lavage can be performed as follows:

a) applying gas positive end-expiratory pressure (PEEP) with a ventilator into a lung section of the mammal at a regulated pressure, preferably from about 4 to 20 cm water;

b) instilling a lavage composition containing dilute surfactant polypeptides in a pharmaceutically acceptable aqueous medium into one or more lobes or sections of the lung; and

c) removing the resulting pulmonary fluid from the lung using short intervals of tracheo-bronchial suction, preferably using a negative pressure of about 20 to 100 mm mercury.

Typically, the PEEP is applied for a preselected time period prior to instilling step (b), preferably up to about 30 minutes, and in addition PEEP is typically applied continuously during steps (b) and (c) and for a preselected time period after removing step (c), preferably up to about 6 hours.

The following examples are intended to illustrate, but not limit, the present invention.

### EXAMPLE 1

### Preparation of Surfactant Protein/Polypeptide

Synthesis of a surfactant polypeptide of the present invention, e.g., KL₄, may be carried out according to a variety of known methods of synthesis. The following procedure is described as exemplary.

Alternatively, the following procedure is also used as described herein. Chemicals and reagents useful in synthesizing batches of surfactant peptides, e.g., batches of KL₄ peptide, include the following:
t-Boc-L-lysine(C1-Z) PAM-resin (t-Boc-L-Lys (C1-Z) (Applied Biosystems, Foster City, Calif.);
a-Boc-ε-(2-Chloro-CBZ)-L-Lysine (Bachem, San Diego, Calif.);
N-Boc-L-Leucine-H₂O (N-Boc-L-Leu; Bachem);
Dichloromethane (DCM; EM Science, Gibbstown, N.J., or Fisher, Pittsburgh, Pa.);
Trifluoroacetic acid (TFA; Halocarbon);
Diisopropylethylamine (DIEA; Aldrich, Milwaukee, Mich.);
N,N-Dimethylformamide (DMF; EM Science, Gibbstown, N.J.);
Dimethylsulfoxide (DMSO; Aldrich);
N-Methylpyrrolidone (NMP; Burdick Jackson, Muskegon, Mich.);
1-Hydroxybenzotriazole hydrate (HOBt; Aldrich);
1,3-Dicyclohexylcarbodiimide (DCC; Aldrich);
Acetic anhydride (Ac₂O; Mallinckrodt, St. Louis, Mo.); and
Hydrogen fluoride (HF; Air Products, Allentown, Pa.)

One means of synthesizing KL₄ peptide (SEQ ID NO:1) is performed on a Coupler 296 Peptide Synthesizer (Vega Biotechnologies, Tucson, Ariz.) using the Merrifield method. A “typical” synthesis is described as follows. Chain elongation was carried out on 100 g of lysine PAM-resin by the procedure described in Table 2 below. All steps except steps 7, 10 and 11 were done automatically.

TABLE 2
Program for a Cycle Using the HOBt Active Ester Procedure

| | | | |
|---|---|---|---|
| Step | Reagent | Time | Volume |

| | | | | |
|---|---|---|---|---|
| 1 | 50% TFA/CH₂Cl₂ | 1 × 2 | min | 1.8 liters |
| 2 | 50% TFA/CH₂Cl₂ | 1 × 20 | min | 1.5 liters |
| 3 | CH₂Cl₂ | 5 × 20 | sec | 1.7 liters |
| 4 | 5% DIEA/CH₂Cl₂ | 1 × 2 | min | 1.7 liters |
| 5 | 5% DIEA/NMP | 1 × 3 | min | 1.7 liters |
| 6 | DMF | 5 × 30 | sec | 1.7 liters |
| 7 | BOC AA-HOBt active | 1 × 39 | min | 1.0 liters |
| | ester | | | |
| 8 | DIEA/DMSO | 1 × 21 | min | 0.5 liters |
| | (195 ml/285 ml) | | | |
| 9 | DMF | 3 × 30 | sec | 1.7 liters |
| 10 | 10%; AC₂O/ | 1 × 8 | min | 2.0 liters |
| | 5% DIEA/NMP | | | |
| 11 | CH₂Cl₂ | 3 × 30 | sec | 1.7 liters |

While the peptide-resin was being deprotected, the appropriate amino acid derivative was being made. The appropriate amino acid was dissolved in one (1) liter of NMP. After a clear solution was obtained, HOBt was added to the solution. When the HOBt was dissolved, DCC was added to the solution. This solution was left stirring for one (1) hour at room temperature. During this one hour of stirring, a by-product formed, dicyclohexylurea (a white precipitate). This by-product was filtered off through a buchner funnel using Whatman's #1 filter paper. The filtrate was then added manually to the contents of the Vega 296 reaction vessel at step No. 7.

The synthesizer was then programmed to stop after the completion of step No. 9. Aliquots of the peptide resin were subjected to the quantitative ninhydrin test of Sarin et al. (Applied Biosystems 431A user manual, Appendix A). The coupling efficiencies were good throughout the entire synthesis. The unreacted peptide resin was acetylated after leucine 12 (cycle 9) and after leucine 5 (cycle 16). After each acetylation, the peptide resin was washed with dichloromethane (see Table 2, step 11).

At the end of the synthesis, the completed peptide resin was deprotected (removal of the Boc group) by completing steps 1-3 of the program (see Table 2). The deprotected peptide resin was then washed with ample volumes of absolute ethanol and dried in vacuo over P₂O₅ The weight of the dried, deprotected peptide resin was 256.48 grams. Since the batch was started with 100 g of t-Boc-Lysine (C1-Z) OCH₂ PAM resin at a substitution of 0.64 mmoles/gram, the load corresponded to 64 mmoles. Subtracting out the initial 100 grams of resin, the weight gain was 156.48 grams. The molecular weight of the nascent protected peptide (excluding the C-terminal lysine anchored onto the resin) was 3011.604 g/mole.

HF Cleavage. The 256.48 gram lot of peptide resin was treated with hydrogen fluoride (HF) in three large aliquots. A Type V HF-Reaction Apparatus from Peninsula Laboratories (Belmont, Calif.) was used for the cleavage of the peptide resin using liquid hydrogen fluoride. The anisole was distilled before use. HF was used without any treatment. Dry ice, isopropanol and liquid nitrogen are required for cooling purposes.

For the first HF, approximately 88 g of the KL₄ peptide resin was placed into the one-liter reaction vessel with a magnetic stir bar. Twenty-five ml of distilled anisole was added to the peptide resin. After the entire system was assembled and leak-tested, HF was condensed into the reaction vessel until the overall level reached about 300 ml. Cleavage of the peptide from the resin was allowed to proceed for one hour at −4° C. Partial removal of HF was done by water aspirator for 1-2 hours. After the 1-2 hours, the rest of the HF was removed by high vacuum (mechanical vacuum pump) for 1-2 hours. The temperature of the reaction vessel remained at −4° C. throughout the HF removal process.

The HF apparatus was then equilibrated to atmospheric pressure and an oily sludge was found at the bottom of the reaction vessel. Cold anhydrous ether (700 ml, prechilled to −20° C.) was added to the contents of the reaction vessel. The resin clumps were triturated with ether using a glass rod. The ether was decanted after the resin settled. The resin was then washed with 500 ml of room temperature anhydrous ether and allowed to stir for about 5 min. The ether was decanted after the resin settled. The resin was washed until it became free-flowing (4-5 total washes). The resin was left in the fume hood to dry overnight.

The resulting dried HF-treated resin was then weighed and stored in the freezer. 1.021 grams of the dried HF-treated resin was removed and extracted with 50 ml of 50% acetic acid/water and allowed to stir for 30 min. The resin was filtered through a coarse sintered glass funnel, and the filtrate was collected in a lyophilizing jar. The filtrate was diluted with approximately 200 ml of water, shell frozen, and placed on the lyophilizer. The one (1) gram of extracted HF-treated resin yielded 569 mg of crude peptide. The following table (Table 3) summarizes the large scale HF treatments of the remaining KL₄ peptide resin. All of the HF-treated resins were stored in the freezer.

TABLE 3

| | Wt. of | Amt. of | Total Volume |
|---|---|---|---|
| HF# | Resin | Anisole | (HF + Anisole + Resin) |
| 1 | 88.07 g | 25 ml | 300 ml |
| 2 | 85.99 g | 25 ml | 300 ml |
| 3 | 79.35 g | 25 ml | 300 ml |

Purification. The peptide was purified using a Dorr-Oliver Model B preparative HPLC (Dorr-Oliver, Inc., Milford, Conn.). This unit was connected to a Linear Model 204 spectrophotometer and Kipp and Zonen dual channel recorder. This preparative HPLC was interfaced with a Waters KL250 Column Module (Waters Associates, Milford, Mass.) containing a radially compressed 10×60 cm cartridge filled with Vydac C₄ support, 15-20 microns, and 300 A pore size (Vydac, Hesperia, Calif.). Solvent “A” consisted of 0.1% HOAc in water, and solvent “B” consisted of 0.1% HOAc in acetonitrile. The flow rate was set at 400 ml/min, the cartridge was compressed to 150-200 psi, and the preparative HPLC system back pressure was at 550-600 psi.

For the first Dorr-Oliver run, 20 g of the HF treated resin from HF#1 was extracted in 500 ml of glacial acetic acid for five minutes. Water (500 ml) was added to the resin/acetic acid mixture. This 50% acetic acid/water solution was stirred for an additional 25 minutes. The resin was filtered off with a coarse sintered glass funnel. The peptide-containing filtrate was saved and loaded onto the Dorr-Oliver. The HPLC gradient used was 1-40% “B” in 45 minutes, then held isocratically for seven minutes. At this point, the percent “B” was increased 1% per minute to a final percentage of 44% (not shown).

Fractions were collected manually and were analyzed by HPLC. All fractions that met a purity of ≧95% were pooled together and stored in a large glass container. This material was subsequently referred to as “BPS #1.” All fractions that had the desired component, but did not meet the 95% or better purity, were collected and later recycled. At least 10 additional preparative HPLC runs were performed on the Dorr-Oliver unit (data not shown).

Reverse Osmosis, Lyophilization. The total volume of BPS #1 was approximately 60 liters. Reverse osmosis was used to concentrate the peptide solution to a final volume of two liters. A Millipore Model 6015 Reverse osmosis Unit with an R75A membrane to retain the peptide was used. The resulting two liters of BPS #1 were filtered through a buchner funnel using two pieces of Whatman #1 filter paper, divided into approximately 11 lyophilizing jars and diluted with equal volumes of water. The lyophilizing jars were shell-frozen and lyophilized. The total weight of dry KL₄ peptide at the end of the procedure was 40.25 g.

Re-lyophilization. It has been found that different lyophilizing conditions (e.g. peptide concentration, composition of solvents to be lyophilized, length of the lyophilization step, shelf temperature, etc.) can result in dried preparations having differing solubility characteristics. It is desirable that the dry KL₄ peptide be soluble in a chloroform: methanol (1:1) solution at 1 mg/ml and ≧90% soluble at 10 mg/ml. If these criteria are not met at the end of the lyophilization step noted above, the peptide can be re-lyophilized.

A typical re-lyophilization is described as follows. Approximately 5 g of peptide is slowly added to two liters of acetonitrile stirring in a glass flask. After approximately one minute, three liters of Milli-Q water is added, followed by 50 ml of acetic acid (final concentration of acetic acid=1%). This is stirred for three days at 37° C., filtered through Whatman #1 filter paper in a buchner funnel, and placed into a lyophilization jar. It is then shell frozen using dry ice and isopropyl alcohol and placed on the lyophilizer. Lyophilization time may vary from three to seven days. The final dry product is then weighed, packaged, and aliquots taken for solubility and chemical analyses.

### EXAMPLE 2

### Preparation of Model Surfactant Mixture

Materials. 1,2-dipalmitoyl phosphatidylcholine (DPPC), 1-palmitoyl, 2-oleoyl phosphatidylglycerol (POPG), and palmitic acid (PA) were obtained from Avanti Polar Lipids Inc. (Birmingham, Ala.). The KL₄ polypeptide with the amino acid sequence KLLLLKLLLLKLLLLKLLLLK (SEQ ID NO:1) was synthesized as described herein or obtained from Discovery Laboratories, Inc., (Doylestown, Pa.). All salts, buffers and organic solvents used were of the highest grade available.

A stock solution of surfactant composition was formulated to contain 40 mg/mL total phospholipid, with a composition based on the following formula:
PL_{T}=total phospholipid=DPPC+POPG
3 DPPC:1 POPG
1 PL_{T}: 0.15 PA:0.027 KL4 peptide.

Using the foregoing formula, surfactant compositions were made that contained varying amounts of palmitic acid (PA) and the KL₄ peptide in 2.5 to 30 mg per mL of total phospholipids (Table 4).

TABLE 4

| Component | 2.5 mg/mL | 10 mg/mL | 30 mg/mL | |
|---|---|---|---|---|
| DPPC | 1.875 mg | 7.5 mg | 22.5 | mg |
| POPG | 0.625 mg | 2.5 | 7.5 | mg |
| PA | 0.375 mg | 1.5 | 4.5 | mg |
| KL₄ Peptide | 0.067 mg | 0.267 | 0.801 | mg |

A Model Surfactant Mixture was made as follows. KL₄ peptide (9 mg), DPPC (225 mg), POPG (75 mg) and PA (45 mg) were dissolved in 2.5 milliliters (ml) of 95% ethanol at 45° C. This solution was then added to 7.5 ml of distilled H₂O at 45° C. with rapid vortexing and 2 ml of 500 mM NaCl, 250 mM Tris-acetate pH 7.2 was added. The resulting milky suspension was stirred at 37° C. for 15 minutes and the ethanol present was then removed by dialysis (Spectrapor 2; 13,000 mol. wt. cutoff) against 100 volumes of 130 mM NaCl, 20 mM Tris-acetate pH 7.2 buffer at 37° C. Dialysis was continued for 48 hours with two changes of the dialysis solution.

In addition, the composition may further comprise a buffer system/suspension having the following composition per mL of finished product (Table 5).

TABLE 5

| Component | Amount per mL |
|---|---|
| Tromethamine, USP | 2.42 mg |
| Glacial acetic acid, USP | quantity sufficient to adjust |
| or NaOH, NF | tromethamine buffer to pH 7.7 |
| NaCl, USP | 7.6 mg |
| Water for injection, USP | Quantity sufficient to 1.0 mL |

This Tham buffered system was prepared essentially as follows. 0.37 ml of Tham solution (tromethamine injection, NDC 0074-1593-04, Abbott Laboratories, North Chicago, Ill.), with the pH adjusted using acetic acid (AR Select, ACS, Mallinckrodt, Paris, Ky.) to a pH of 7.2±0.5, was admixed with 0.33 ml saline (0.9% sodium chloride injection, USP, Abbott Laboratories) and 0.30 ml water (sterile water for injection, USP, Abbott Laboratories). The solution was sterile-filtered.

### References

Amaro, A., Inhale Therapeutics Report, 14, 2001.
Cochrane, C G, et al., Am. J. Resp. and Crit. Care Med., Vol. 163:139, 2001.
Enhorning, et al., Am. J. Respir. Crit. Care Med., 151:554-556, 1995.
Freide, M., et al., Anal. Biochem., 211(1):117-122, 1993.
Glasser, et al., J. Biol. Chem., 263:10326, 1988.
Ilowite, et al., Am. Rev. Respir. Dis., 136:1445-1449, 1987.
Janoff, A., In: INFLAMMATION: BASIC PRINCIPLES AND CLINICAL CORRELATES, Gallin, J. I., et al., eds, 803-814, Raven Press, New York, 1988.
Jobe, et al., Am. Rev. Resp. Dis., 136:1032, 1987.
Kharasch, V. S., et al., Am. Rev. Respir. Dis., 144:909-913, 1991.
King, et al., Am. J. Physiol., 223:715-726, 1972.
Laube, et al., Chest, 95:822-830, 1989.
Lee, C. T., et al., New England J. of Med., 304:192-196, 1981.
Maa, Y. F., Pharm. Dev. Technol., 2(3):213-223, 1997.
Maa, Y. F., et al., Pharm. Res., 15(5):768-775, 1998.
Master, K., SPRAY DRYING HANDBOOK, 5^{th} edition, J. Wiley & Sons, New York, 1991.
Martin, F. J., In: SPECIALIZED DRUG DELIVERY SYSTEMS-MANUFACTURING AND PRODUCTION TECHNOLOGY, P. Tyle, ed., Marcel Dekker, New York, pp. 267-316, 1990.
Mayer, L. D., et al., Biochim. Biophys. Acta, 857:123-126, 1986.
Mayer, L. D., et al., Canc. Res., 49:5922-5930, 1989.
Meienhofer, J., In: HORMONAL PROTEINS AND PEPTIDES, Vol. 2, p. 46, Academic Press, New York, 1983.
Niven, R. W., Modulated Drug Therapy with Inhalation Aerosols, in A. J.
Hickey, ed., Marcel Dekker, New York, 1992.
Notter, et al., Clin. Perinatology, 14:433-79, 1987.
Olson, F., et al., Biochim. Biophys. Acta, 557:9-23, 1979.
Puchell, E., et al., Eur. J. Clin. Invest., 15:389-394, 1985.
Revak, et al, Am. Rev. Respir. Dis., 134:1258-1265, 1986.
Robertson, Lung, 158:57-68, 1980.
Sarbolouki, M. N., Toliat, T., PDA J. Pharm. Sci. Technol., 52 (1):23-27, 1998.
Schroder, E., Kubke, K., In: THE PEPTIDES, Vol. 1, Academic Press, New York, 1965.
Steward, J. M., Young, J. D., In: SOLID PHASE PEPTIDE SYNTHESIS, W.H. Freeman Co., San Francisco, 1969.
Szoka, F. Jr., et al., Ann. Rev. Biophys. Bioeng., 9:467-508, 1980

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed:

1. A method of treating or preventing asthma in a mammal comprising administering to the mammal a composition comprising an effective amount of phospholipid and an isolated lung surfactant polypeptide.

2. The method of claim 1, wherein the lung surfactant polypeptide comprises a polypeptide having between 10-60 amino acid residues and an amino acid sequence of alternating hydrophobic and hydrophilic amino acid residue regions represented by the formula (ZₐU_{b})_{c}Z_{d},
wherein Z is a hydrophilic amino acid residue, U is a hydrophobic amino acid residue, a is an integer with an average value of 1-5, b is an integer with an average value of 3-20, c is an integer of about 1 to about 10, and d is an integer of about 0 to about 3.

3. The method of claim 2, wherein Z is histidine, lysine, arginine, aspartic acid, glutamic acid, 5-hydroxylysine, 4-hydroxyproline or 3-hydroxyproline.

4. The method of claim 2, wherein U is valine, isoleucine, leucine, cysteine, tyrosine, phenylalanine, and/or an α-aminoaliphatic carboxylic acid, such as α-aminobutanoic acid, α-aminopentanoic acid, α-amino-2-methylpropanoic acid, or α-aminohexanoic acid.

5. The method of claim 2, wherein U is α-aminobutanoic acid, α-aminopentanoic acid, α-amino-2-methylpropanoic acid, or α-aminohexanoic acid.

6. The method of claim 1, wherein the lung surfactant polypeptide comprises amino acid sequence:
(SEQ ID NO:1)
KLLLLKLLLLKLLLLKLLLLK,
(SEQ ID NO:2)
KLLLLLLLLKLLLLLLLLKLL,
(SEQ ID NO:3)
KKLLLLLLLKKLLLLLLLKKL,
(SEQ ID NO:4)
DLLLLDLLLLDLLLLDLLLLD;
(SEQ ID NO:5)
RLLLLRLLLLRLLLLRLLLLR;
(SEQ ID NO:6)
RLLLLLLLLRLLLLLLLLRLL;
(SEQ ID NO:7)
RRLLLLLLLRRLLLLLLLRRL,
(SEQ ID NO:8)
RLLLLCLLLRLLLLCLLLR,
(SEQ ID NO:9)
RLLLLCLLLRLLLLCLLLRLL,
(SEQ ID NO:10)
RLLLLCLLLRLLLLCLLLRLLLLCLLLR;
or
(SEQ ID NO:11)
(Xa)(Xb)LLLL(Xa)LLLL(Xa)(Xb)LLLL(Xa)LLL(Xa)(Xb)
wherein each Xa is separately selected from lysine or arginine, and each Xb is separately selected from aspartic acid or glutamic acid.

7. The method of claim 1, wherein the lung surfactant polypeptide is

| | |
|---|---|
| KLLLLKLLLLKLLLLKLLLLK. | (SEQ ID NO:1) |

8. The method of claim 1, wherein the lung surfactant polypeptide comprises amino acid sequence SEQ ID NO:11, SEQ ID NO:12, SEQ ID NO:13, SEQ ID NO:14, SEQ ID NO:15, SEQ ID NO:16 or SEQ ID NO:17.

9. The method of claim 1, wherein the composition comprises an amount of lung surfactant polypeptide that is at about 0.1 to 10 percent of the amount of phospholipid.

10. The method of claim 1, wherein the composition comprises about 50 to about 95 dry weight percent phospholipids.

11. The method of claim 1, wherein the phospholipid comprises phosphatidylcholines, phospatidylglycerols, phosphatidylserines, phosphatidylinositols, phosphatidic acids, or phosphatidylethanolamines.

12. The method of claim 1, wherein the phospholipid comprises dipalmitoyl phosphatidylcholine, dilauryl phosphatidylcholine, dimyristoyl phosphatidylcholine, distearoyl phosphatidylcholine, diphytanoyl phosphatidylcholine, nonadecanoyl phosphatidylcholine, arachidoyl phosphatidylcholine, dioleoyl phosphatidylcholine, dipalmitoleoyl phosphatidylcholine, linoleoyl phosphatidylcholine, dipalmitoyl phosphatidylethanolamine, dioleoylphosphatidyl-ethanolamine, dioleoyl phosphatidylglycerol, palmitoyloleoyl phosphatidylglycerol, distearoylphosphatidylserine, soybean lecithin, egg yolk lecithin, sphingomyelin, phosphatidylserine, phosphatidylglycerol, phosphatidyl inositol, diphosphatidyl glycerol, phosphatidyl-ethanolamine, or phosphatidic acid.

13. The method of claim 1, wherein the phospholipid comprises dipalmitoyl phosphatidylcholine and palmitoyl, oleoyl phosphatidyl glycerol, in a mole ratio of about 4:1 to about 2:1.

14. The method of claim 1, wherein the composition further comprises about 2 to about 25 dry weight percent of the spreading agent.

15. The method of claim 14, wherein the spreading agent is a fatty acid or fatty alcohol having a fatty acyl chain length of at least 10 carbon atoms.

16. The method of claim 14, wherein the spreading agent further includes tyloxapol.

17. The method of claim 1, wherein the composition is administered by inhalation.

18. The method of claim 1, wherein the composition is administered as a liquid bolus to pulmonary tissues.

19. The method of claim 1, wherein the composition is a liquid composition.

20. The method of claim 1, wherein the composition is a dry composition.

21. The method of claim 1, wherein the composition comprises aerosol particles.

22. The method of claim 21, wherein the aerosol particles have a mass median aerodynamic diameter of about 1 μm to about 5 μm.

23. A method of treating or preventing asthma in a mammal comprising administering to the mammal a composition comprising phospholipid and a lung surfactant polypeptide having any one of amino acid sequences SEQ ID NO:1-18.

24. The method of claim 23, wherein the composition comprises an amount of lung-surfactant polypeptide that comprises about 0.1 to 10 percent of the phospholipid.

25. The method of claim 23, wherein the composition comprises about 50 to about 95 dry weight percent phospholipids.

26. The method of claim 23, wherein the phospholipid comprises phosphatidylcholines, phospatidylglycerols, phosphatidylserines, phosphstidylinositols, phosphatidic acids, or phosphatidylethanolamines.

27. The method of claim 23, wherein the phospholipid comprises dipalmitoyl phosphatidylcholine, dilauryl phosphatidylcholine, dimyristoyl phosphatidylcholine, distearoyl phosphatidylcholine, diphytanoyl phosphatidylcholine, nonadecanoyl phosphatidylcholine, arachidoyl phosphatidylcholine, dioleoyl phosphatidylcholine, dipalmitoleoyl phosphatidylcholine, linoleoyl phosphatidylcholine, dipalmitoyl phosphatidylethanolamine, dioleoylphosphatidyl-ethanolamine, dioleoyl phosphatidylglycerol, palmitoyloleoyl phosphatidylglycerol, distearoylphosphatidylserine, soybean lecithin, egg yolk lecithin, sphingomyelin, phosphatidylserine, phosphatidylglycerol, phosphatidyl inositol, diphosphatidyl glycerol, phosphatidyl-ethanolamine, or phosphatidic acid.

28. The method of claim 23, wherein the phospholipid comprises dipalmitoyl phosphatidylcholine and palmitoyl, oleoyl phosphatidyl glycerol, in a mole ratio of about 4:1 to about 2:1.

29. The method of claim 23, wherein the composition further comprises about 2 to about 25 dry weight percent of the spreading agent.

30. The method of claim 29, wherein the spreading agent is a fatty acid or fatty alcohol having a fatty acyl chain length of at least 10 carbon atoms.

31. The method of claim 29, wherein the spreading agent further includes tyloxapol.

32. The method of claim 23, wherein the composition is administered by inhalation.

33. The method of claim 23, wherein the composition is administered as a liquid bolus to pulmonary tissues.

34. The method of claim 23, wherein the composition is a liquid composition.

35. The method of claim 23, wherein the composition is a dry composition.

36. The method of claim 23, wherein the composition comprises aerosol particles having a mass median aerodynamic diameter of about 1 μm to about 5 μm.

37. A composition comprising a pharmaceutically acceptable carrier, and a therapeutically effective amount of a lung surfactant polypeptide and a bronchodilator.

38. The composition of claim 37, wherein the bronchodilator is albuterol, levalbuterol, terbutaline, salmeterol, or formoterol.

39. The composition of claim 37, wherein the composition is formulated for pulmonary delivery.

40. A method of treating or preventing asthma in a mammal comprising administering to the mammal an effective amount of phospholipid combined with an isolated KL₄ lung surfactant polypeptide having amino acid sequence SEQ ID NO:1.


SEQUENCE LISTING (18 sequences)

SEQ ID NO: 1 - protein, 21 aa, Artificial Sequence
  A synthetic polypeptide.
KLLLLKLLLLKLLLLKLLLLK

SEQ ID NO: 2 - protein, 21 aa, Artificial Sequence
  A synthetic polypeptide.
KLLLLLLLLKLLLLLLLLKLL

SEQ ID NO: 3 - protein, 21 aa, Artificial Sequence
  A synthetic polypeptide.
KKLLLLLLLKKLLLLLLLKKL

SEQ ID NO: 4 - protein, 21 aa, Artificial Sequence
  A synthetic polypeptide.
DLLLLDLLLLDLLLLDLLLLD

SEQ ID NO: 5 - protein, 21 aa, Artificial Sequence
  A synthetic polypeptide.
RLLLLRLLLLRLLLLRLLLLR

SEQ ID NO: 6 - protein, 21 aa, Artificial Sequence
  A synthetic polypeptide.
RLLLLLLLLRLLLLLLLLRLL

SEQ ID NO: 7 - protein, 21 aa, Artificial Sequence
  A synthetic polypeptide.
RRLLLLLLLRRLLLLLLLRRL

SEQ ID NO: 8 - protein, 19 aa, Artificial Sequence
  A synthetic polypeptide.
RLLLLCLLLRLLLLCLLLR

SEQ ID NO: 9 - protein, 21 aa, Artificial Sequence
  A synthetic polypeptide.
RLLLLCLLLRLLLLCLLLRLL

SEQ ID NO: 10 - protein, 28 aa, Artificial Sequence
  A synthetic polypeptide.
RLLLLCLLLRLLLLCLLLRLLLLCLLLR

SEQ ID NO: 11 - protein, 21 aa, Artificial Sequence
  A synthetic polypeptide.
HLLLLHLLLLHLLLLHLLLLH

SEQ ID NO: 12 - protein, 248 aa, Homo sapiens
MWLCPLALNLILMAASGAVCEVKDVCVGSPGIPGTPGSHGLPGRHGRDGLKGDLGPPGPMGPPGEMPCPPGNDGLPGAPGIPGECGEKGEPGERGPPGLRAHLDEELQATLHDFRHQILQTRGALSLQGSIMTVGEKVFSSNGQSITFDAIQEACARAGGRIAVPRNPEENEAIASFVKKYNTYAYVGLTEGPSPGDFRYSDGTPVNYTNWYRGEPAGRGKEQCVEMYTDGQWNDRNCLYSRLTICEF

SEQ ID NO: 13 - protein, 248 aa, Homo sapiens [sequence omitted]

SEQ ID NO: 14 - protein, 381 aa, Homo sapiens
MAESHLLQWLLLLLPTLCGPGTAAWTTSSLACAQGPEFWCQSLEQALQCRALGHCLQEVWGHVGADDLCQECEDIVHILNKMAKEAIFQDTMRKFLEQECNVLPLKLLMPQCNQVLDDYFPLVIDYFQNQIDSNGICMHLGLCKSRQPEPEQEPGMSDPLPKPLRDPLPDPLLDKLVLPVLPGALQARPGPHTQDLSEQQFPIPLPYCWLCRALIKRIQAMIPKGALRVAVAQVCRVVPLVAGGICQCLAERYSVILLDTLLGRMLPQLVCRLVLRCSMDDSAGPRSPTGEWLPRDSECHLCMSVTTQAGNSSEQAIPQAMLQACVGSWLDREKCKQFVEQHTPQLLTLVPRGWDAHTTCQALGVCGTMSSPLQCIHSPDL

SEQ ID NO: 15 - protein, 197 aa, Homo sapiens
MDVGSKEVLMESPPDYSAAPRGRFGIPCCPVHLKRLLIVVVVVVLIVVVIVGALLMGLHMSQKHTEMVLEMSIGAPEAQQRLALSEHLVTTATFSIGSTGLVVYDYQQLLIAYKPAPGTCCYIMKIAPESIPSLEALNRKVHNFQMECSLQAKPAVPTSKLGQAEGRDAGSAPSGGDPAFLGMAVNTLCGEVPLYYI

SEQ ID NO: 16 - protein, 374 aa, Homo sapiens
MLPFLSMLVLLVQPLGNLGAEMKSLSQRSVPNTCTLVMCSPTENGLPGRDGRDGREGPRGEKGDPGLPGPMGLSGLQGPTGPVGPKGENGSAGEPGPKGERGLSGPPGLPGIPGPAGKEGPSGKQGNIGPQGKPGPKGEAGPKGEVGAPGMQGSTGAKGSTGPKGERGAPGVQGAPGNAGAAGPAGPAGPQGAPGSRGPPGLKGDRGVPGDRGIKGESGLPDSAALRQQMEALKGKLQRLEVAFSHYQKAALFPDGRSVGDKIFRTADSEKPFEDAQEMCKQAGGQLASPRSATENAAIQQLITAHNKAAFLSMTDVGTEGKFTYPTGEPLVYSNWAPGEPNNNGGAENCVEIFTNGQWNDKACGEQRLVICEF

SEQ ID NO: 17 - protein, 9 aa, Artificial Sequence
  MOD_RES 1: succinyl
  MOD_RES 9: amide
  A synthetic polypeptide.
LLEKLLQWK

SEQ ID NO: 18 - protein, 23 aa, Artificial Sequence
  A synthetic polypeptide.
  SITE 1, 7, 12, 18, 22: Xaa is Lys or Arg.
  SITE 2, 13, 23: Xaa is Asp or Glu.
XXLLLLXLLLLXXLLLLXLLLXX